(19) United States
(12) Patent Application Publication
(10) Pub. No.: US 2024/0261401 A1
(43) Pub. Date: Aug. 8, 2024

(54) ENGINEERED IMMUNE CELLS, COMPOSITIONS AND METHODS THEREOF

(71) Applicant: iCell Gene Therapeutics Inc., Stony Brook, NY (US)

(72) Inventors: Yupo MA, Stony Brook, NY (US); Kevin CHEN, Stony Brook, NY (US); Kevin PINZ, Stony Brook, NY (US); Xun JIANG, Stony Brook, NY (US); Masayuki WADA, Stony Brook, NY (US)

(21) Appl. No.: 17/928,414

(22) PCT Filed: May 26, 2021
(86) PCT No.: PCT/US2021/034271

§ 371 Date: Nov. 29, 2022

Related U.S. Application Data
(60) Provisional application No. 63/032,138, filed on May 29, 2020.

(51) Int. Cl. A61K 39/00; A61P 35/02; C07K 14/54; C07K 14/715; C12N 5/0783

(52) U.S. Cl. CPC A61K 39/4611; A61K 39/4613; A61P 35/02; C07K 14/5418; C07K 14/5443; C07K 14/7155; C12N 5/0636; C12N 5/0646; A61K 2239/48; C07K 2319/02; C07K 2319/03; C12N 2510/00

(57) ABSTRACT

The present invention relates to compositions and methods relating to an engineering cell with enhancer moiety in treating the subject with cancers and infection diseases. The engineering cells include T cells, NK cells, T NK cells, gamma delta (γδ) T cells and MR1-R T cells.

### BACKGROUND

T cells, a type of lymphocyte, play a central role in cell-mediated immunity. They are distinguished from other lymphocytes, such as B cells and natural killer cells (NK cells), by the presence of a T-cell receptor (TCR) on the cell surface.

NK cells are a type of innate immune cell that play an important role in preventing viral and/or bacterial infections and tumor formation. However, NK cells have a short half-life of approximately 7 days. Extension of the NK cell half-life could greatly enhance its functional activity, including responding to viral and/or bacterial infections and treating cancers.

COVID-19 is a newly identified virus that consists of a single-stranded positive-sense RNA coronavirus. Patients infected with COVID-19 often develop respiratory problems after infection resulting in death in some patients. As of today, there is no specific treatment for this newly identified virus.

### SUMMARY OF THE INVENTION

In one embodiment, the present disclosure provides an engineered NK cell having IL-15 or IL-15/IL-15sushi or IL-15/IL-15sushi anchor.

In another embodiment, the present disclosure provides a method of reducing the number of viral infected cells or viral particles in a host in need thereof comprising administering a composition comprising (i) an engineered or modified NK or T cell and (ii) an IL-7, IL-15, IL-/IL-15sushi, IL-15/IL-15 sushi anchor, CCL-119 or CCL-21 to said host in need thereof.

In yet another embodiment, a method for ex vivo expansion of NK cells and T cells comprising: 1) isolation of NK or T cells; 2) introduction of at least one of enhancers selected from a group of IL-7, IL-15, IL-15sushi, IL-15/IL-15anchor, CCL-19 (CCL19) and CCL-21 (CCL21) and 3) expansion of NK or T cells.

In one embodiment, the present disclosure provides methods for treating patients infected with viruses, bacteria, fungi and/or parasites by administering an engineered immune cell.

In another embodiment, the present disclosure provides methods for treating patients with viral infections including, but not limited to, herpes simplex virus (HSV), Epstein-Barr viruses (EBV), varicella Zoster virus (VZV), cytomegalovirus (CMV), human papilloma virus (HPV), human immunodeficiency virus (HIV) and coronavirus.

In a further embodiment, the present disclosure provides methods for treating coronaviruses including, but not limited, middle east respiratory syndrome (MERS), severe acute respiratory syndrome (SARS) and COVID-19.

In one embodiment, the present disclosure provides methods for treating a patient with COVID-19 by administering an engineered immune cell.

In another embodiment, the present disclosure provides methods for treating a patient with cancers by administering an engineered immune cell.

### BRIEF DESCRIPTION OF DRAWINGS

FIG. 1A. A schematic showing a secreting IL-15/IL-15sushi construct with a rituximab epitope. The construct consists of a SFFV promoter driving the expression of an IL-15/IL-15sushi and a rituximab safety switch linked by a P2A self-cleavage peptide. Upon cleavage of this P2A peptide, the enhancer IL-15/IL-15sushi protein and rituximab safety switch protein are split. Secreting IL-15/IL-15sushi (enhancer) comprises a leader sequence and IL-15/IL-15sushi fusion protein. Rituximab safety protein comprises a leader sequence, two copies of rituximab epitopes, a hinge (H) region, and a transmembrane domain (TM). The self-cleavage peptides of the construct may include, but is not limited to, P2A, T2A, F2A and E2A. The secreting protein (s) of the construct may also include, but is not limited to, IL-15/IL-15sushi, IL-15, IL-21, IL-18, IL-7 and IL-12. The secreting enhancer, such as IL-15/IL-15sushi enhances T or NK cell expansion and persistency. The soluble IL-15/IL-15sushi fusion is stable and functions as an unexpected and powerful immunomodulatory for T/NK cells and their neighbor tumor immune response cells. The soluble IL-15/IL-15sushi fusion is also able to enhance T/NK cell persistency and stimulate T/NK cell functions of anti-pathogen or anti-tumor activities. The soluble IL-15/IL-15sushi fusion also provides vaccine-like effects by reprogramming the body's immune system to fight infections and cancers.

FIG. 1B. Schematic diagram of secreting IL-15/IL-15sushi expressed in T and NK cells. The soluble IL-15/IL-15sushi fusion is stable and functions as an unexpected and powerful immunomodulatory for T/NK cells and their neighbor tumor immune response cells. The soluble IL-15/IL-15sushi fusion enhances T/NK cell persistency and stimulates T/NK cell functions in anti-pathogen or anti-tumor activities. The soluble IL-15/IL-15sushi fusion provides vaccine-like effects by reprogramming the body's immune system to fight infections and cancers through stimulating immune cell expansion and their functions.

FIG. 2A. A schematic showing an IL-15/IL_15sushi anchor. A) the construct consists of a SFFV promoter driving the expression of an IL-15/IL-15sushi anchor (also called anchor). The IL-15/IL-15sushi anchor is composed of a signal peptide fused to IL-15 and linked to sushi domain of IL-15 alpha receptor via a 26-amino acid poly-proline linker, two copies of rituximab epitopes (stop), hinge (H) region and a transmembrane domain (TM). IL-15/IL-15sushi is anchored on the surface of T or NK cells, which results in enhancing NK and T cell expansion and persistency.

FIG. 2B. IL-15/IL-15sushi anchor on the surface of T or NK cells. IL-15/IL-15sushi is anchored on the surface of T or NK cells, which results in enhancing NK and T cell expansion and persistency.

FIG. 3A. Steps for generation and preparation of irradiated genetically modified K562 cells as feeder cells for cord blood NK cell expansion.

FIG. 3B. Steps for generation and expansion of CAR-transduced natural killer (NK) cells from umbilical cord blood by co-culture with irradiated genetically modified K562 cells (feeder cells).

FIG. 4A. Evaluation of persistence of IL15/IL15sushi transduced NK cells in xenograft mouse model on day 60. IL15/IL15sushi transduced NK cells infused to SCID mice. Peripheral blood was collected from individual injected mice and were labeled using human CD56- and human CD45 antibodies to detect the presence of infused-NK cells. The persistence of 15/IL15suhi transduced NK cells in collected peripheral blood was determined by flow cytometry analysis. NK cells were undetectable in control mice starting about day 7-10 days while mice transduced with IL-15/IL-15sushi had detect NK cells at day 60 post-infusion. Left panels are negative controls, Right panels are IL15/IL15sushi-transduced NK cells infused mice.

FIG. 4B. Evaluation of persistence of IL15/IL15sushi transduced NK cells in xenograft mouse model on day 95. IL15/IL15sushi transduced NK cells infused to SCID mice. Peripheral blood was collected form individual injected mice and were labeled using human CD56- and human CD45 antibodies to detect the presence of infused-NK cells. The persistence of 15/IL15suhi transduced NK cells in collected peripheral blood was determined by flow cytometry analysis. NK cells were undetectable in control mice starting about day 7-10 days while mice transduced with IL-15/IL-15sushi had detect NK cells at Day 60 post-infusion. Left panels are negative controls, Right panels are IL15/IL15sushi-transduced NK cells infused mice.

FIG. 5. Detection of secreting IL-15 in supernatants by ELISA in NK cells transduced with IL-15/IL-15sushi constructs. Sorted IL-15/IL-15sushi NK92 cells and wild-type control NK-92 cells were cultured in separate wells for 72 hours. Supernatant was collected and subjected to ELISA on 96-well plates precoated with IL-15 antibody. Following manufacturer's (Boster) directions, colorimetric results obtained on a plate reader were compared to a standard curve (A) generated with human IL-15 to determine concentration of IL-15 in the supernatants (B).

FIG. 6. Summary of the effect of secreting IL-15/IL-15sushi and IL-15/IL-15sushi anchor NK92 cells and non-transduced neighboring NK92 cells by flow cytometry analysis. GFP+NK92 cells showed significantly prolonged survival in co-cultured in the absence of IL-2 when co-cultured with IL-15/IL-15sushi-transduced NK-92 compared to −IL-15/IL-15sushi anchor −NK92. These studies indicate that secreting IL-15/IL-15sushi complexes have a profound effect on CAR cells and their neighboring non-transduced cells. In contrast, IL-15/IL-15sushi anchor had a similar effect on transduced cells compared to secreting IL-15/IL-15sushi but its effect on neighboring non-transduced cells was limited.

FIG. 7A. Evaluation of persistence of IL-7-IL-15/IL-15sushi anchor T cells in xenograft mouse model on day 1, 5 and 9. Human umbilical cord blood lymphocytes were isolated and transduced with IL-7-IL-15/IL-15sushi anchor construct. This construct expresses secreting IL-7 and IL-15/IL-15sushi anchor on cell surface. About 1.6×10⁵ IL-7-IL-15/IL-15sushi anchor transduced cells/mouse were injected intravenously (mouse tail). Peripheral blood was collected form individual injected mouse and was labeled using anti-human CD56- and CD3 antibodies to detect the presence of infused T cells. The persistence of IL-7-IL-15/IL-15sushi anchor T cells in collected peripheral blood was determined by flow cytometry analysis.

FIG. 7B. Evaluation of persistence of IL-7-IL-15/IL-15sushi anchor T cells in xenograft mouse model on day 14, 21 and 28. Human umbilical cord blood lymphocytes were isolated and transduced with IL-7-IL-15/IL-15sushi anchor construct. This construct expresses secreting IL-7 and IL-15/IL-15sushi anchor on cell surface. About 1.6×10⁵ IL-7-IL-15/IL-15sushi anchor transduced cells/mouse were injected intravenously (mouse tail). Peripheral blood was collected from individual injected mice and was labeled using anti-human CD56- and CD3 antibodies to detect the presence of infused T cells. The persistence of IL-7-IL-15/IL-15sushi anchor T cells in collected peripheral blood was determined by flow cytometry analysis.

FIG. 7C. Evaluation of persistence of IL-7-IL-15/IL-15sushi anchor T cells in xenograft mouse model on day 35, 42 and 49. Human umbilical cord blood lymphocytes were isolated and transduced with IL-7-IL-15/IL-15sushi anchor construct. This construct expresses secreting IL-7 and IL-15/IL-15sushi anchor on the cell surface. About 1.6×10⁵ IL-7-IL-15/IL-15sushi anchor transduced cells/mouse were injected intravenously (mouse tail). Peripheral blood was collected from individual injected mice and was labeled using anti-human CD56- and CD3 antibodies to detect the presence of infused T cells. The persistence of IL-7-IL-15/IL-15sushi anchor T cells in collected peripheral blood was determined by flow cytometry analysis.

FIG. 7D. Evaluation of persistence of IL-7-IL-15/IL-15sushi anchor T cells in xenograft mouse model on day 56, 63 and 70. Human umbilical cord blood lymphocytes were isolated and transduced with IL-7-IL-15/IL-15sushi anchor construct. This construct expresses secreting IL-7 and IL-15/IL-15sushi anchor on the cell surface. About 1.6×10⁵ IL-7-IL-15/IL-15sushi anchor transduced cells/mouse were injected intravenously (mouse tail). Peripheral blood was collected from individual injected mice and was labeled using anti-human CD56- and CD3 antibodies to detect the presence of infused T cells. The persistence of IL-7-IL-15/IL-15sushi anchor T cells in collected peripheral blood was determined by flow cytometry analysis.

FIG. 8. A schematic showing a secreting IL-15/IL-15sushi construct with immunoglobulin FAB light chain tag and rituximab epitopes (also called 4LV-Q-IL-15R). The construct consists a SFFV promoter driving the expression of a rituximab safety switch and an IL-15/IL-15sushi linked by a P2A self-cleavage peptide. Upon cleavage of the P2A peptide. enhancer, rituximab safety switch protein and IL-15/IL-15sushi are separated. Secreting IL-15/IL-15sushi (enhancer) comprises a leader sequence and IL-15/IL-15sushi fusion protein. Rituximab safety protein comprises a leader sequence, an immunoglobulin FAB light chain tag, two copies of rituximab epitopes, a hinge (H) region, a transmembrane domain (TM). The peptide self-cleavage peptides of the construct may include, but is not limited to, P2A, T2A, F2A and E2A. The secreting protein (s) of the construct may also include, but is not limited to, IL-2, IL-15/IL-15sushi, IL-15, IL-21, IL-18, IL-7 and IL-12. The secreting enhancer, such as IL-15/IL-15sushi enhances T or NK cell expansion and persistency. The soluble IL-15/IL-15sushi fusion is stable and functions as an unexpected and powerful immunomodulatory for T/NK cells and their neighbor tumor immune response cells. The soluble IL-15/IL-15sushi fusion are stable and enhances T/NK cell persistency and stimulates T/NK cell functions in anti-pathogen or anti-tumor activities. The soluble IL-15/IL-15sushi fusion provides vaccine-like effects by reprogramming body's immune system to fight infections and cancers.

FIG. 9. Expression of 4LV-Q-IL-15R construct secreting IL-15/IL-15sushi with immunoglobulin FAB light tag and rituximab epitopes on T cells. Buffy coat cells were activated 3 days with anti-CD3 antibody. Cells were transduced with either control vector (left), or 4LV-Q-IL-15 lentiviral supernatant. The 4LV-Q-IL-15 bears a secreting IL-15/IL-15sushi co-expressing with an immunoglobulin FAB light chain tag (upper panel) and rituximab epitopes (low panel). 4LV-Q-IL-15R transduced T cells with lentiviral supernatant are shown (right). After 3 days of incubation, cells were harvested and labeled for flow cytometry.

FIG. 10. A schematic showing GL-Q-7xp-TM construct containing IL-15/IL-15 anchor with immunoglobulin FAB light chain tag (GL), rituximab epitopes and secreting IL-7. The construct consists a SFFV promoter driving the expression of a rituximab safety switch and secreting IL-7 and an IL-15/IL-15sushi anchor linked by P2A and T2A self-cleavage peptides, respectively. Upon cleavage of the P2A and T2A peptides, enhancers, rituximab safety switch protein and IL-7 and IL-15/IL-15suhi anchor are separated. Rituximab safety protein comprises a leader sequence, an immunoglobulin FAB light chain tag, two copies of rituximab epitopes, a hinge (H) region, a transmembrane domain (TM). Secreting IL-7 (enhancer) comprises a leader sequence and IL-7 protein. The IL-15/IL-15sushi anchor is composed of a signal peptide fused to IL-15 and linked to sushi domain of IL-15 alpha receptor via a 26-amino acid poly-proline linker, hinge (H) region and a transmembrane domain (TM). IL-15/IL-15sushi is anchored on the surface of T or NK cells, which results in enhancing NK and T cell expansion and persistency. Secreting IL-7 enhances IL-15/IL-15sushi anchor's functions in immune cell expansion and persistency.

FIG. 11. A schematic showing GL-Q-IL-15R-TM construct containing IL-15/IL-15 anchor with immunoglobulin FAB light chain tag (GL), rituximab epitopes and secreting IL-15/IL-15sushi. The construct consists of a SFFV promoter driving the expression of a rituximab safety switch and secreting IL-15/IL-15sushi and an IL-15/IL-15sushi anchor linked by P2A and T2A self-cleavage peptides, respectively. Upon cleavage of the P2A and T2A peptides, enhancers, rituximab safety switch protein and IL-15/IL-15sushi (or IL-15) and IL-15/IL-15suhi anchor are separated. Rituximab safety protein comprises a leader sequence, an immunoglobulin FAB light chain tag, two copies of rituximab epitopes, a hinge (H) region, a transmembrane domain (TM). Secreting IL-15/IL-15sushi (enhancer) comprises a leader sequence and IL-IL-15/IL-15sushi protein. The IL-15/IL-15sushi anchor is composed of a signal peptide fused to IL-15 and linked to sushi domain of IL-15 alpha receptor via a 26-amino acid poly-proline linker, hinge (H) region and a transmembrane domain (TM). IL-15/IL-15sushi is anchored on the surface of T or NK cells, which results in enhancing NK and T cell expansion and persistency. Secreting IL-15/IL-15sushi or IL-15 enhances IL-15/IL-15sushi anchor's functions in immune cell expansion and persistency.

FIG. 12A. CD19 based CARs deplete Reh cells in vivo and co-expression of IL-15/IL-15sushi strongly enhances anti-tumor response. Mice were injected with Reh tumor cells (0.5×10⁶ cells/mouse) expressing luciferase on Day 1. On Day 3, IVIS was conducted to assay the appearance of Reh cells. On Day 4, control T-cells, CD19b CAR, and CD19b-IL15/IL15sushi CAR T-cells were injected (˜7.5×10⁶ total cells/mouse) and on day 6 through 22, IVIS imaging was conducted to assay semi-quantitative assessment of tumor burden and subsequent tumor depletion and control of cell growth by T-cells. Here, both CART treatments demonstrated similar efficacy, with the IL-15/IL-15sushi armored CAR demonstrating comparable or better control of the Reh tumor growth when compared to standard CART19 cells.

FIG. 12B. Line graph plotting IVIS values (estimation of tumor burden) against time for the treatment cohorts. As the tumor burden rises within the control group, both CART groups show steady maintenance of tumor suppression with significantly decreased tumor counts as measured by statistical analysis.

FIG. 12C. Comparison CD19b-CAR-T (CART19) vs CD19b-IL-15/IL15sushi CAR-T against REH cells over long term. Similar experimental scheme with identical IVIS methodology as above; however, mice were followed until signs of tumor relapse were seen. Here, after day 30, we observed that aggressive Reh tumor relapse began to occur in standard CART19 treated mice. Clusters of tumor (indicated by red regions on the IVIS imaged mice) were seen in most CART19 mice, with a single CD19b-IL-15/IL-15sushi CART treated mice also showing tumor growth by day 22. However, after day 30, all CART19 mice showed signs of severe tumor relapse, while CD19b-IL-15/IL-15sushi CART treated mice showed no sign of tumor. Even the relapsed mouse on day 22 was absolved of its tumor by day 32, signifying that CD19b-IL-15/IL-15sushi CART cells were still in effective circulation.

FIG. 12D. IL-15/IL-15sushi armor is able to prevent disease relapse after standard CAR T fails. Line graph summarizing IVIS trend values estimating tumor growth over time for each treatment cohort. Past day 30, the tumor burden for the standard CD19b CAR (CART19) treated mice rises precipitously, resulting in highly significant increases in tumor burden compared to the CD19b-IL-15/IL-15sushi armored CART treatment group which remained largely tumor free. Values are displayed for both views of the mice (ventral and dorsal image acquisition views).

FIG. 13A. Overall summary of mice blood data (summarized persistency of CAR T cells in mice). The overall persistence of T cells in mouse blood from the model in FIG. 42C was assayed at survival endpoints and screened by flow cytometry using CD3 antibody for bulk T cell populations. To further dissect the persistency results of the CD19b-IL-15/IL-15sushi armored CAR, the collection of mouse blood is necessary to reveal the presence of durability of the engrafted human cells. Overall, we found by flow cytometry analysis that there was a higher average count of T cells in the armored CAR cohorts when compared to the standard CART19 groups. Control group T cells remained at baseline as expected due to minimal stimulation from circulating in vivo tumor.

FIG. 13B. Further dissection of engrafted CAR T phenotype characteristics. Mouse blood characteristics from FIG. 42B between CD19b (CART19) and CD19b-IL-15/IL-15sushi CAR T cells were further compared by analyzing the CD4 and CD8 population subsets. In general, there were a higher amount of CD3+ cells in the armored CAR cohort, correlating with increased persistency, a higher average of CD8+ cells within the CD3+ effector T cell population in the armored CAR cohort, and increased ability of the armored CAR T cells to bear the central memory immune-phenotype, correlating with improved immune-surveillance.

FIG. 14A. A schematic showing a CAR 19-Q-XX CAR. A CD19 CAR equipped with a cytokine complex, IL-15/IL-15sushi and a chemokine, CCL19. The construct consists a SFFV promoter driving the expression of a CAR and a secreting cytokine linked by a P2A peptide, a secreting chemokine separated by a T2A. Upon cleavage of P2A and T2A peptides, CD19-Q-XX CAR splits to a CAR, a cytokine complex, IL-15/IL15-sushi, and a chemokine, CCL19. CAR has scFv, hinge region (H), transmembrane domain (TM), costimulatory domain (including, but not limited to CD28 or 4-1BB) and intracellular signaling, CD3 zeta chain. Immune cells used for this study can include, but not limited to, T cells, NK cells, NKT cells and NK-92 cells. Wherein hinge region bears a safe switch, two CD20 mimotopes (also called Q), which enable CAR T cells fast and efficient eradication by the Rituximab (RTX).

FIG. 14B. CD19b-XX-CAR-T-cells cells exhibit significant anti-tumor activity, and greater persistence than CD19b-IL-15/IL-15sushi CAR T cells, in xenogeneic mouse model. NSG mice were sublethally irradiated and intravenously injected with ˜0.3×10⁶ luciferase-expressing REH cells to induce measurable tumor formation. Starting 7 days after injection of tumor cells, mice were intravenously injected with a course of 0.3×10⁶ CD19b-IL-15/IL-15sushi (three center mice), or CD19b-XX (three right mice) CAR T cells or vector control T cells (three left mice). On days 6 (before T cell injection), 9 (after T cell injection), 14, 20, 29, 34 and 45, mice were injected subcutaneously with RediJect D-Luciferin and subjected to IVIS imaging.

FIG. 14C. NSG mice injected with REH tumor cells survive significantly longer when treated with CD19b-XXCAR T cells compared to mice treated with CD19b-IL-15/IL-15 sushi CAR T cells. Survival curve. Following the IVIS imaging experiments previously described in FIG. 5, mice were observed every day for symptoms of severe illness, and were sacrificed once movement was greatly impaired. All control mice were sacrificed by Day 27 (not shown), and all mice treated with CD19b-IL-15/IL-15 sushi CAR T were sacrificed by Day 53 (red line). In contrast, all mice treated with CD19b-XXCAR T cells survived until at least Day 60 (blue line). This difference between the groups was shown to be significant by the Mantel-Cox test (0.0246) and the Gehan-Breslow-Wilcoxon test (P=0.0339).

FIGS. 15A and 15B. Transduction of activated human T cells with CD19b-RTX-TM-CAR-lentiviral vector and evaluation of expression levels of surface CD19b-CAR- and RTX on T cells for infusion of mice. CD19b-RTX-TM construct contains CD19 CAR co-expressing IL-15/IL-15 anchor (TM) with rituximab epitopes (RTX, also called Q). The construct consists of a SFFV promoter driving the expression of a rituximab safety switch and an IL-15/IL-15sushi anchor linked by a self-cleavage peptides. IL-15/IL-15sushi is anchored on the surface of T or NK cells, which results in enhancing NK and T cell expansion and persistency. Surface expression of CD19b-CAR and rituximab (RTX; circled in green on bottom panels) on CD19b-RTX-TM-CAR-virus transduced T-cells were determined using flow cytometry analysis (FIG. 15A). The upper panels show the expression level of CD19b-RTX-TM-CAR on T cells (red dots circled in blue) after transduction of CD19b-RTX-TM-CAR-virus in cells. The bottom panels show the expression levels of recombinant RTX protein (safety switch) on T cells (red dots circled in green) using CD34 antibody after transduction of CAR-virus in cells. CD34 antibody can recognize the part of RTX epitope. Luciferase-expressing REH cells (1×10⁶ cells) were injected intravenously (day 1) in mice 24 hours after sub-lethal irradiation (2.0 Gy). On day 5, 10×10⁶ of CD19b-RTX-TM-CAR expressing T-cells or control T-cells were intravenously injected into the mice. Images of dorsal sides and ventral sides of mice were taken (FIG. 15B).

FIGS. 16A and 16B. Transduction of CD19b-IL15/IL15sushi-RTX-TM-CAR viruses into T cells and evaluation of its expression levels on T cells for infusion of mice. CD19b-IL15/IL15sushi-RTX-TM construct contains CD19 CAR co-expressing secreted IL-15/IL-15 and IL-15/IL-15 anchor (TM) with rituximab epitopes (RTX, also called Q) separated by a self-cleavage peptide. The construct consists of a SFFV promoter driving the expression of CD19 CAR, a rituximab safety switch, secreted IL-15/IL-15sushi and IL-15/IL-15sushi anchor linked by self-cleavage peptides. IL-15/IL-15/IL-15sushi is secreted from the transduced T and NK cells and IL-15/IL-15 anchor is anchored on the surface of T or NK cells. Both secreted IL-15/IL-15/IL-15sushi and IL-15/IL-15 anchor involve synergistically enhancing NK and T cell expansion and persistency. Surface expression of CD19b-CAR and rituximab (RTX; circled in green on bottom panels) on CD19b-IL15/IL15sushi-RTX-TM-CAR-virus transduced T-cells were determined using flow cytometry analysis (FIG. 16A). On day 5, 10×10⁶ of CD19b-IL15/IL15sushi-RTX-TM-CAR expressing T-cells or control T-cells were intravenously injected into the mice. Images of dorsal sides and ventral sides of mice were taken (FIG. 16B).

FIGS. 17A and 17B. Transduction of CD19b-RTX-7-TM-CAR-virus into T cells and evaluation of its expression levels for infusion of mice.

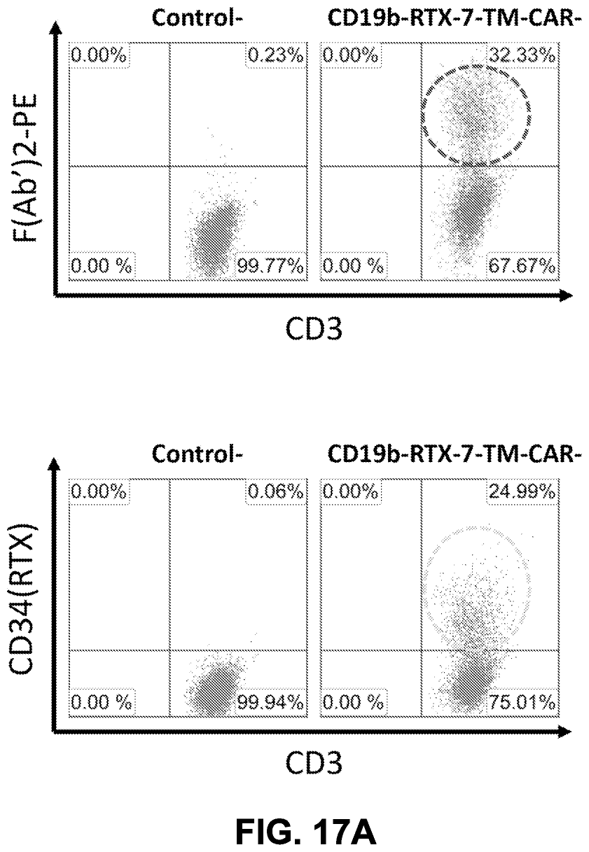

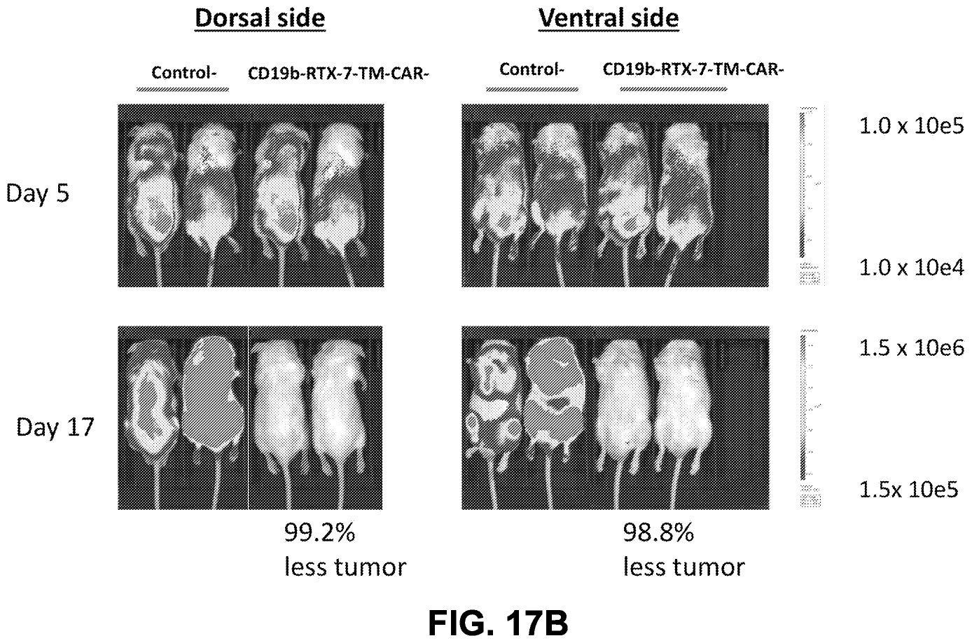

CD19b-RTX-7-TM-construct contains CD19 CAR co-expressing secreted IL-7 and IL-15/IL-15 anchor (TM) with rituximab epitopes (RTX, also called Q) separated by a self-cleavage peptide. The construct consists of a SFFV promoter driving the expression of CD19 CAR, a rituximab safety switch, secreted IL-7 and IL-15/IL-15sushi anchor linked by self-cleavage peptides. IL-7 is secreted from the transduced T and NK cells and IL-15/IL-15 anchor is anchored on the surface of T or NK cells. Both secreted IL-7 and IL-15/IL-15 anchor involve synergistically enhancing NK and T cell expansion and persistency. Surface expression of CD19b-CAR and rituximab (RTX; circled in green on bottom panels) on CD19b-RTX-7-TM-CAR-virus transduced T-cells were determined using flow cytometry analysis (FIG. 17A). On day 5, 10×10⁶ of CD19b-RTX-7-TM-CAR expressing T-cells or control T-cells were intravenously injected into the mice. Images of dorsal sides and ventral sides of mice were taken (FIG. 17B).

### DETAILED DESCRIPTION

The disclosure provides description of engineered immune cells, compositions, methods of manufacture and use thereof.

Immune cells or immunomodulatory cells including, but not limited to, T cells, macrophage, NK cells and NK T cells have been used for treatment of infectious diseases and cancers. NK cells in particularly have been shown to effectively treat infectious diseases and residual cancers. However, the potency is not sufficient to combat infectious diseases and cancers, partly due to their short biologic half-life and limit of immune functions. Accordingly, the present invention is to provide engineered NK cells co-expressing with an immune function-enhancing factor have a high immunity-inducing activity against infectious diseases and cancers. The present disclosure also provides methods to generate engineer NK cells that are able to secrete an immune function-enhancing factor that reprograms the immune system to combat infectious diseases and cancers.

In certain embodiments, NK cells are derived from human peripheral blood mononuclear cells (PBMC), leukapheresis products (PBSC), human embryonic stem cells (hESCs), induced pluripotent stem cells (iPSCs), bone marrow, or umbilical cord blood.

The potential disadvantages of using NK cells in a cellular therapy include a lack of persistency that may reduce long-term efficacy.

In one embodiment, the present disclosure comprises a method of modified NK cells with long-lived or long persistency in vivo for treating a disease. Surprisingly, it is found that NK cells co-expressing IL-15/IL-15sushi or IL-15/IL-15 sushi anchor can extend survival for a long period of time.

IL-15 is a pleiotropic cytokine that is associated with a huge range of immunology and plays an important role in both adaptive and innate immunity.

### Objects to be Solved by the Invention

Allogeneic or autologous NK cells induce a rapid immune response but disappear relatively rapidly from the circulation due to their limited lifespan and poor persistency.

The NK cell is an ideal platform against tumors or infections if these cells can persist a relatively long period of time. However, the life expectancy of NK cells in vivo is very short, with a lifespan of one or two weeks. Ideally, the NK cell persistency should be one or two months to be considered adequate for therapy.

IL-15 functions through a trimeric IL-15R complex, which contains a high affinity binding α-chain (IL-15 Rα) and the common IL-2R β- and γ-chains. IL-15 secreting from a cell binds to IL-15 Rα associated with IL-15 receptor β- and γ-chains on the surface of cells.

Allogeneic or autologous NK cells induce a rapid immune response but disappear relatively rapidly from the circulation due to their limited lifespan

Constitutive expression of a high level of IL-15 in mice could cause leukemia (Fehniger et al, J Exp Med. 2001 Jan. 15; 193(2):219-31). IL-15Rα (full-length of IL-15 receptor alpha subunit) accelerates leukemia development in T cells when constitutive co-expression with IL-15 (Sato et al, Blood. 2011 Apr. 14; 117).

The inventors disclose the method to improve immune cell functions while preventing tumor formation.

### Inventive Steps to Solve the Objects

A 65 amino acid sequence of the extracellular portion of IL-15Rα, called sushi domain involves the binding of IL-15. It has been known that the cytoplasmic domain of IL-15 receptor a chain is critical for normal IL-15Rα functions.

The invention discloses a method of fusing IL-15 to the sushi domain instead of the full of IL-15Rα to form an IL-15/IL-15sushi fusion. In further disclosures, the signaling cytoplasmic domain of IL-15Rα is not included in the IL-15/IL-15sushi fusion. In a further disclosure, IL-15/IL-15sushi fusion is expressed and anchored on the surface of a cell, which is called IL-15/IL-15sushi anchor.

In accordance with the present disclosure, it was surprisingly found that cells expressing secreted IL-15/IL-15sushi do not observe leukemia formation in human clinical trials after more than two-year observation.

In some embodiments, IL-15/IL-15sushi fusion is expressed as a protein precursor secreted from a cell.

A protein precursor, is an inactive protein that can be turned into an active form by post-translational modification.

IL-15 is responsible for vaccine-like effects by promotion and proliferation of T cells and innate cells including NK cells. IL-15 has a very short biological half-life of about 2 hours. Our addition of the sushi domain to form an IL-15/IL-15sushi complex increases this half-life of IL-15, up to ten-fold leading to longer persistency.

In some embodiments, it is preferred that a low level and longer biologic half-life of IL-15 is preferred in vivo.

It was surprisingly found that only picogram quantities of IL-15/IL-15sushi were produced by immune cells transduced with IL-15/IL-15sushi without evidence of autonomous growth in vitro or leukemic transformation in human clinical trials after at least a 2-year observation.

In accordance with the present disclosure, the inventors have also found that immune cells transduced with secreted IL-15/IL-15sushi are superior in persistency and immunity-inducing effect to the conventional immune cells in vivo.

In order to increase this efficiency, a different leader sequence, IL-2 is used to replace the wild-type IL-15 leader sequence to achieve higher levels of secretion. Furthermore, it is known that IL-15 has a short biological half-life. Furthermore, it is known that IL-15 has a short biological half-life. The sushi domain is incorporated to increase IL-15 half-life up to ten-fold by forming an IL-15/IL-15sushi complex, leading to longer persistency.

Prior to the art, it has been found that IL-15Rα (full-length of IL-15 receptor alpha subunit) accelerates T cell leukemia development when constitutively co-expressed with IL-15 (Sato et al, Blood. 2011 Apr. 14; 117) in transgenic mouse models (Sato et al, Blood. 2011 Apr. 14; 117).

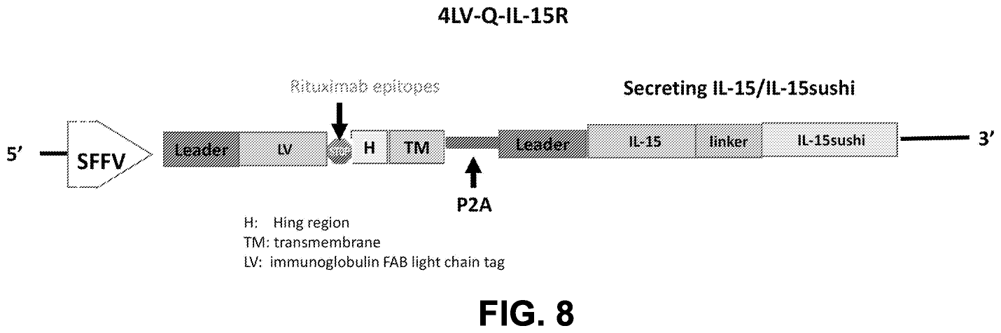

The present disclosure describes an IL-15/IL-15sushi anchor having IL-15/IL-15 sushi expression on the surface of an immunomodulatory cell to enhance its functions. This IL-15/IL-15 sushi anchor comprises of a 65 amino acid segment of the extracellular portion of IL-15 sushi domain involving the binding of IL-15. The invention lacks the cytoplasmic functional domain and most of the extracellular domain of IL-15Rα, in order to avoid leukemic formation. However, this omission is compensated for by the incorporation into the design of either secreted IL-7 or IL-15 or IL-15/IL-15sushi. This secretion can be easily controlled using a safety switch (FIGS. 8, 10 and 11), thereby turning off expression in adverse conditions.

In some embodiments, the invention discloses a method of establishment of a NK cell platform for a universal therapy with improved persistency of NK cells and their killing activities using secreting IL-15/IL-15sushi fusion. NK cells co-expressing secretory IL-15/IL-15sushi can be used as a universal platform for treatment of a variety of diseases.

In one embodiment, the present disclosure provides an engineered cell expressing IL-15/IL-15sushi anchor.

In further embodiment, the extension of NK cell persistency can be achieved by co-expressing the IL-15/IL-15sushi anchor.

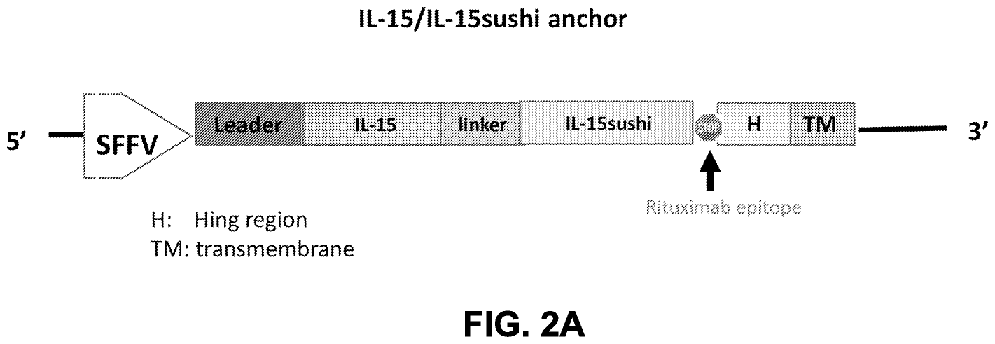

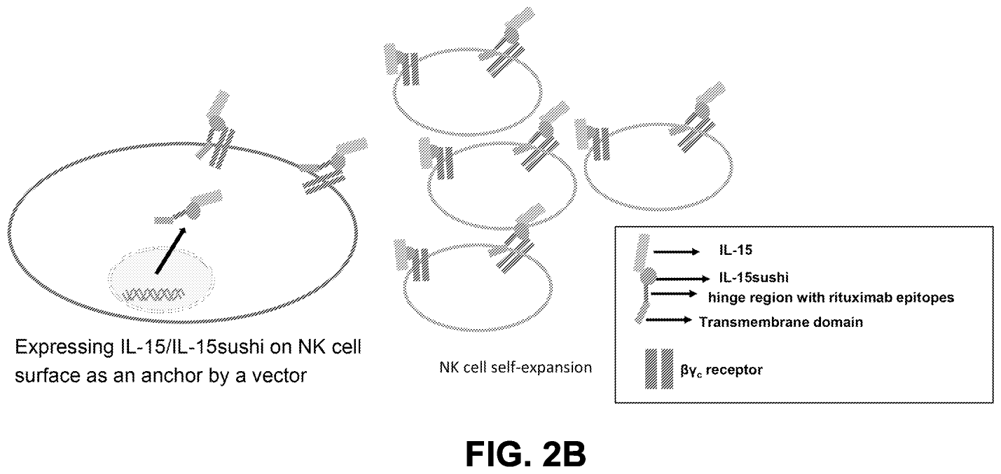

In one embodiment, the present also disclosure provides an IL-15/IL-15sushi anchor having an IL-15/IL-15 sushi, a signal peptide, a hinge region and a transmembrane domain (see FIG. 2A).

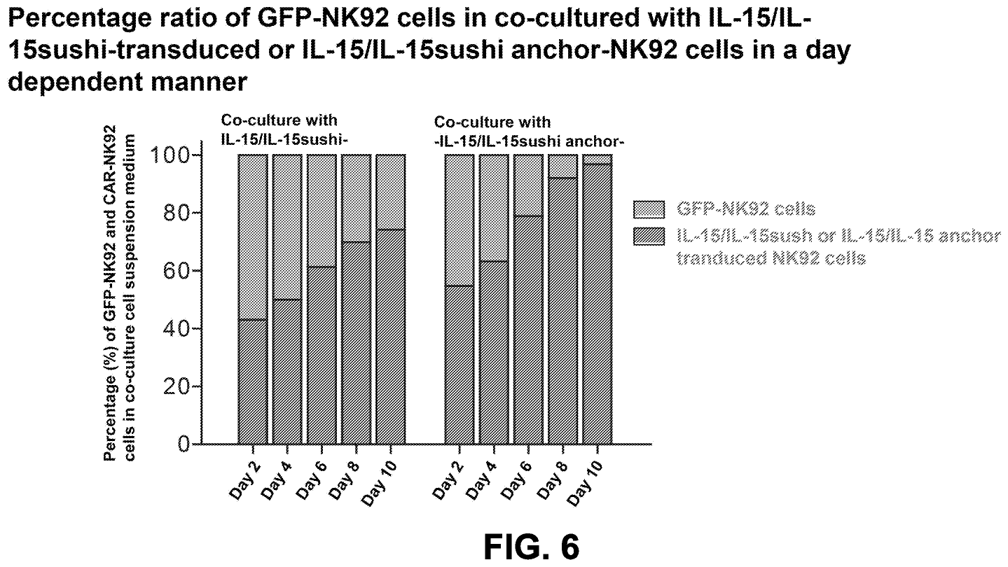

Without wishing to be bound by theory, it is believed that expressing IL-15/IL-15sushi anchor in an immune cell does not cause tumor formation as IL-15/Il-15sushi anchor lacks the entire cytoplasmic domain of IL-15 receptor alpha, which has been shown to be critical for its normal function (Wu et al, Blood. 2008 Dec. 1; 112(12): 4411-4419). It is surprisingly found that IL-15/IL-15sushi anchor lacking the entire critical cytoplasmic functional domain is still able to enhance the immune cell persistency (FIG. 6).

### Inventive Steps to Solve the Objects Related to Engineered Cell Trafficking and Migration

Viruses utilize various strategies to evade or delay the cytokine response, which allow them to replicate in the host. Tumors produce a microenvironment to suppress immune cell migration and trafficking to tumor sites. Enhancing trafficking and migration of immune cells is critical for their functions in response to infected and neoplastic cells.

In one embodiment, the present invention provides a method of engineering cells to secrete chemokines involving the recruitment of T cells, NK cells and dendritic cells to infected or tumor tissues. Without wishing to be bound by theory, it is believed that expressing CCL-19 or CCL-21, or both, recruits T cells, B cells, NK cells and dendritic cells to infected or tumor tissues.

Both chemokine (C-C motif) ligand 19 (CCL-19) and chemokine (C-C motif) ligand 21 (CCL-21) are cytokines belonging to the CC chemokine family.

In further embodiments, the enhancement of NK or T cell persistency and trafficking can be achieved by co-expressing the IL-15/IL-15sushi and CCL-19.

In further embodiment, the enhancement of NK or T cell persistency and trafficking can also be achieved by co-expressing the IL-15/IL-15sushi and CCL-21.

In further embodiments, the enhancement of NK or T cell persistency and trafficking can be achieved by co-expressing the IL-15 and CCL-19.

In further embodiment, the enhancement of NK or T cell persistency and trafficking can also be achieved by co-expressing the IL-15 and CCL-21

In further embodiments, the enhancement of NK or T cell persistency and trafficking can be achieved by co-expressing the IL-15/IL-15sushi anchor and CCL-19

In further embodiment, the enhancement of T or NK cell persistency and trafficking can also be achieved by co-expressing the IL-15/IL-15sushi anchor and CCL-21

A “signal peptide” includes a peptide sequence that directs the transport and localization of the peptide and any attached polypeptide within a cell, e.g. to a certain cell organelle (such as the endoplasmic reticulum) and/or the cell surface.

The signal peptide is a peptide of any secreted or transmembrane protein that directs the transport of the polypeptide of the disclosure to the cell membrane and cell surface, and provides correct localization of the polypeptide of the present disclosure. In particular, the signal peptide of the present disclosure directs the polypeptide of the present disclosure to the cellular membrane, wherein the extracellular portion of the polypeptide is displayed on the cell surface, the transmembrane portion spans the plasma membrane, and the active domain is in the cytoplasmic portion, or interior of the cell.

In one embodiment, the signal peptide is cleaved after passage through the endoplasmic reticulum (ER), i.e. is a cleavable signal peptide. In an embodiment, the signal peptide is human protein of type I, II, III, or IV. In an embodiment, the signal peptide includes an immunoglobulin heavy chain signal peptide.

The hinge sequence may be obtained including, for example, from any suitable sequence from any genus, including human or a part thereof. Such hinge regions are known in the art. In one embodiment, the hinge region includes the hinge region of a human protein including CD-8 alpha, CD28, 4-1BB, OX40, CD3-zeta, T cell receptor α or β chain, a CD3 zeta chain, CD28, CD38ε, CD45, CD4, CD5, CD8, CD9, CD16, CD22, CD33, CD37, CD64, CD80, CD86, CD134, CD137, ICOS, hemagglutinin (HA) of influenza virus, glycosylphosphatidylinositol (GPI)-anchored protein, CD154 and functional derivatives thereof, and combinations thereof.

In one embodiment, the hinge region includes the CD8a hinge region.

In one embodiment, the hinge region includes the HA hinge region.

In some embodiments, the hinge region includes one selected from, but is not limited to, immunoglobulin (e.g. IgG1, IgG2, IgG3, IgG4, and IgD).

In some embodiments, the hinge region can be excluded in the IL-15/IL-15sushi anchor.

The transmembrane domain includes a hydrophobic polypeptide that spans the cellular membrane. In particular, the transmembrane domain spans from one side of a cell membrane (extracellular) through to the other side of the cell membrane (intracellular or cytoplasmic).

The transmembrane domain may be in the form of an alpha helix or a beta barrel, or combinations thereof. The transmembrane domain may include a polytopic protein, which has many transmembrane segments, each alpha-helical, beta sheets, or combinations thereof.

The transmembrane sequence may be obtained including, for example, from any suitable sequence from any genus, including human or a part thereof. Such transmembrane regions are known in the art. In one embodiment, the transmembrane region includes the transmembrane region of a human protein including a T-cell receptor α or β chain, a CD3 zeta chain, CD28, CD3ε, CD45, CD4, CD5, CD8, CD9, CD16, CD22, CD33, CD37, CD64, CD80, CD86, CD134, CD137, ICOS, CD154, hemagglutinin (HA) of influenza virus, and functional derivatives thereof, and combinations thereof.

In one embodiment, the transmembrane region includes the CD8a transmembrane region.

In one embodiment, the transmembrane region includes the HA transmembrane region.

In some embodiments, NK cells co-expressing IL-15/IL-15sushi or IL-15/IL-15sushi anchor can be scaled up and used as an off-the-shelf product.

In some embodiments, NK cells co-expressing both IL-15/IL-15sushi and IL-15/IL-15sushi anchor can be scaled up and used as an off-the-shelf product. In such embodiments, NK cells comprising the enhancer are expressed in a single polypeptide molecule having a high efficiency peptide cleavage sites including, but not limited to, P2A, T2A, F2A and E2A. In a further embodiment, NK cells comprising the enhancer are expressed in a single open reading frame (ORF) under the control of a strong promoter.

In some embodiments, NK cells co-expressing both IL-7 and IL-15/IL-15sushi anchor can be scaled up and used as an off-the-shelf product. In such embodiments, NK cells comprising the enhancer are expressed in a single polypeptide molecule having a high efficiency peptide cleavage sites including, but not limited to, P2A, T2A, F2A and E2A. In a further embodiment, NK cells comprising the enhancer are expressed in a single open reading frame (ORF) under the control of a strong promoter.

Examples of high efficiency cleavage sites include porcine teschovirus-1 2A (P2A), FMDV 2A (abbreviated herein as F2A); equine rhinitis A virus (ERAV) 2A (E2A); and Thoscaasigna virus 2A (T2A), cytoplasmic polyhedrosis virus 2A (BmCPV2A) and flacherie Virus 2A (BmIFV2A), or a combination thereof. In a preferred embodiment, the high efficiency cleavage site is P2A. High efficiency cleavage sites are described in Kim JH, Lee S-R, Li L-H, Park H-J, Park J-H, Lee KY, et al. (2011) High Cleavage Efficiency of a 2A Peptide Derived from Porcine Teschovirus-1 in Human Cell Lines, Zebrafish and Mice. PLOS ONE 6(4): e18556, the contents of which are incorporated herein by reference.

The expression vector may be a bicistronic or multicistronic expression vector. Bicistronic or multicistronic expression vectors may include (1) multiple promoters fused to each of the open reading frames; (2) insertion of splicing signals between genes; fusion of genes whose expressions are driven by a single promoter; (3) insertion of proteolytic cleavage sites between genes (self-cleavage peptide); and (iv) insertion of internal ribosomal entry sites (IRESs) between genes.

In one embodiment, NK cells co-expressing IL-15/IL-15 sushi or IL-15/IL-15sushi anchor are capable of continuing supportive cytokine signaling, which is critical to their survival post-infusion in a patient.

In one embodiment, NK cells co-expressing IL-7 or IL-15/IL-15sushi anchor are capable of continuing supportive cytokine signaling, which is critical to their survival post-infusion in a patient.

In further embodiment, the extension of NK cell survival can be achieved by co-expressing a cytokine selected from a group of IL-7, IL-15, IL-15/IL-15 anchor, IL-15/IL-15RA, IL-12, IL-18 and IL-21.

Surprisingly, it was found that an immune cell co-expressing IL-15/IL-15sushi in human clinical trials revealed a significant elevation of CD8+ T cells and NK cells associated with increased anti-tumor activity and reduced disease relapses.

In some embodiments, IL-15 can be an IL-15N72D mutant and fused to the soluble domain of IL-15Rα (sushi) to form stable complexes in solution, and this complex exhibits increased biological activity compared to the non-complexed IL-15. The Mutant IL-15N72D can increase IL-15 biological activity (US20120177595 A1).

In some embodiments, a NK cell is packed with different immune defense mechanisms that: 1) alter NK cell responses to infections or tumors by mounting attacks on the targeted cells; 2) enhance NK persistency; 3) reprogram body's immune system to combat infectious diseases or cancers,

In some embodiments, a NK cell expresses at least either a cytokine(s) and/or chemokine(s). Co-expressing cytokines in a NK cell can be selected from a group of cytokines including, but not limited to: IL-15/IL-15sushi, IL-15/IL-15sush anchor, IL-2, IL-4, IL-7, IL-10, IL-12, IL-18, IL-21, GM-CSF, and TGF-β. Co-expressing chemokines in a NK cell can also be selected from a group of chemokines including, but limited to: CCL2, CCL3, CCL4, CCL5, CCL7, CCL8, CCL19, CXCL1, CXCL2, CXCL9, CXCL10, or CXCL12 or CCL-21.

In some embodiments, NK cells co-express IL-15/IL-15 anchor with at least one cytokine selected from a group of cytokines including, but not limited to, IL-15, IL-15/IL-15sushi, IL-2, IL-4, IL-7, IL-10, IL-12, IL-18, IL-21, GM-CSF, and TGF-β.

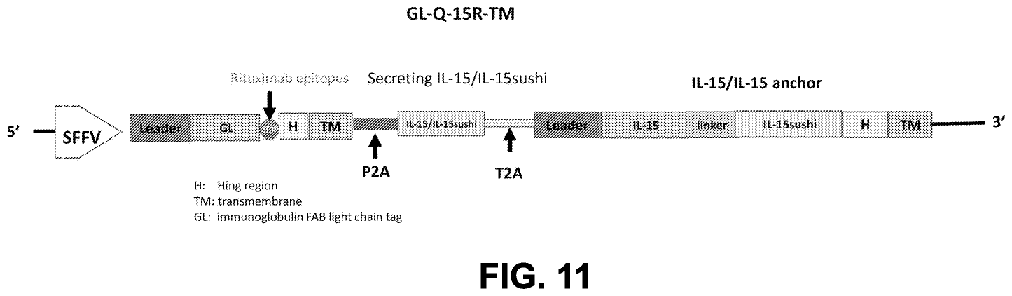

In some embodiments, an engineered cell co-expresses IL-15/IL-15 anchor with IL-15sushi (FIG. 11).

In some embodiments, an engineered cell co-expresses IL-15/IL-15 anchor with IL-15 (FIG. 11).

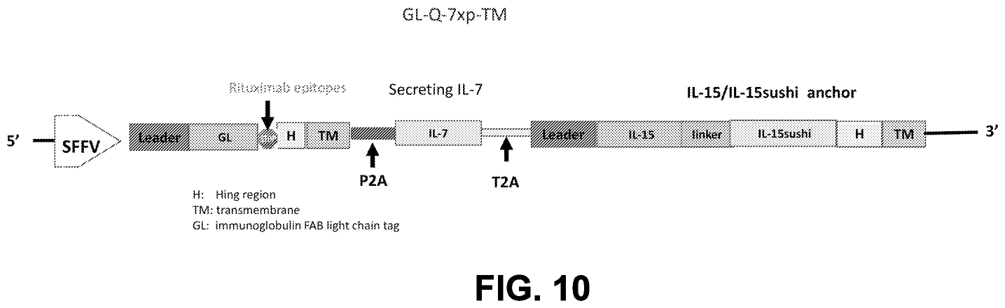

In some embodiments, an engineered cell co-expresses IL-15/IL-15 anchor with IL-7 (FIG. 10).

### Sources of Cells

The engineered cells may be obtained from peripheral blood, cord blood, bone marrow, tumor infiltrating lymphocytes, lymph node tissue, or thymus tissue. The host cells may include placental cells, embryonic stem cells, induced pluripotent stem cells, or hematopoietic stem cells. The cells may be obtained from humans, monkeys, chimpanzees, dogs, cats, mice, rats, and transgenic species thereof. The cells may be obtained from established cell lines.

The above cells may be obtained by any known means. The cells may be autologous, syngeneic, allogeneic, or xenogeneic to the recipient of the engineered cells.

The term “autologous” refer to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenic ally.

The term “xenogeneic” refers to a graft derived from an animal of a different species.

The term “syngeneic” refers to an extremely close genetic similarity or identity especially with respect to antigens or immunological reactions. Syngeneic systems include for example, models in which organs and cells (e.g. cancer cells and their non-cancerous counterparts) come from the same individual, and/or models in which the organs and cells come from different individual animals that are of the same inbred strain.

In certain embodiments. T and NK cells are derived from human peripheral blood mononuclear cells (PBMC), leukapheresis products (PBSC), human embryonic stem cells (hESCs), induced pluripotent stem cells (iPSCs), bone marrow, or umbilical cord blood.

The potential disadvantages of using NK cells as therapy include a lack of persistency that may reduce long-term efficacy.

In some embodiments, engineered cells can immune cells or non-immune cells. Non-immune cells, for instance, can be red blood cells as a carrier to carry cytokines or chemokines to the infected and cancer tissues.

In accordance with the present disclosure, red blood cells as a carrier provide a readily available cell to be engineered to contain at least one cytokine or chemokine selecting from a group of cytokines or chemokines including, but not limited to, IL-15, IL-15/IL-15sush, IL-15/IL-15RA (full length of IL-15 receptor α), IL-15/IL-15 anchor, IL-2, IL-7, IL-12, IL-18, IL-21, CCL2, CCL3, CCL4, CCL5, CCL7, CCL8, CCL19, CXCL1, CXCL2, CXCL9, CXCL10, CXCL12 and CCL-21 polypeptide disclosed

In an embodiment, the engineered cells include immunoregulatory cells.

Engineered immunoregulatory cells include T-cells, such as CD4 T-cells (Helper T-cells), CD8 T-cells (Cytotoxic T-cells, CTLs), and memory T cells or memory stem cell T cells. In another embodiment, T-cells include Natural Killer T-cells (NK T-cells) and gamma delta (γδ) T cells.

In an embodiment, immunoregulatory cells can be derived from embryonic stem cells or induced pluripotent stem cells (IPS cells)

In an embodiment, the engineered cell includes Natural Killer cells. Natural killer cells are well known in the art. In one embodiment, natural killer cells include cell lines, such as NK-92 cells. Further examples of NK cell lines include NKG, YT, NK-YS, HANK-1, YTS cells, and NKL cells.

NK cells mediate anti-tumor effects without the risk of GvHD and are short-lived relative to T-cells. Accordingly, NK cells would be exhausted shortly after destroying targeted cells, decreasing the need for an inducible suicide gene on a construct that would ablate the modified cells.

In accordance with the present disclosure, it was surprisingly found that NK cells provide a readily available cell to be engineered to contain at least one cytokine selecting from a group of cytokines including IL-15, IL-15/IL-15sush, IL-15/IL-15RA (full length of IL-15 receptor α), IL-15/IL-15 anchor, IL-2, IL-7, IL-12, IL-18 and IL-21 polypeptide disclosed herein.

Allogeneic or autologous NK cells induce a rapid immune response but disappear relatively rapidly from the circulation due to their limited lifespan. Thus, applicants surprisingly discovered that there is reduced concern of persisting side effects using NK cell-based therapy.

According to one aspect of the present invention, NK cells can be transfected with cytokine polynucleotides and expanded in accordance to the present invention. NK cells can be derived from cord blood, peripheral blood, iPS cells and embryonic stem cells. According to one aspect of the present invention, NK-92 cells may be expanded and transfected with cytokine polynucleotides. NK-92 is a continuously growing cell line that has features and characteristics of natural killer (NK) cells (Arai, Meagher et al. 2008). NK-92 cell line is IL-2 dependent and has been proven to be safe (Arai, Meagher et al. 2008) and feasible. A pure population of NK-92 carrying the cytokine polynucleotide of interest may be obtained by sorting.

In some embodiments, the engineered cell includes an inducible suicide gene (“safety switch”) or a combination of safety switches, which may be assembled on a vector, such as, without limiting, a retroviral vector, lentiviral vector, adenoviral vector or plasmid. Introduction of a “safety switch” greatly increases safety profile. The “safety switch” may be an inducible suicide gene, such as, without limiting, caspase 9 gene, thymidine kinase, cytosine deaminase (CD) or cytochrome P450. Other safety switches for elimination of unwanted modified NK or T cells involve expression of CD20 or CD20 epitopes or CD52 or CD19 or truncated epidermal growth factor receptor in T cells. All possible safety switches have been contemplated and are embodied in the present invention.

In one embodiment, the engineered cell includes a rituximab safety switch for elimination of unwanted modified immune cells. In a further embodiment, two rituximab binding sequences are incorporated to the hinge region of IL-15/IL-15sushi anchor.

In one embodiment, the engineered cell co-expresses a rituximab epitope expression construct with IL-15/IL-15sushi through a peptide cleavage sequence selected from one of group of P2A, T2A, E2A and F2A. In a further embodiment, the rituximab epitope expression construct comprises of a signal peptide, two epitope domains of rituximab, CD8α hinge region and CD8α transmembrane domain.

Rituximab, originating as a CD20 targeted chimeric antibody, was developed by IDEC pharmaceuticals for treatment of malignancy.

### MR T Cells, an Immunoregulatory Cell

Mucosal associated invariant T cells (MAIT cells) consist of a small subset of T cells in the immune system that exhibit innate. MAIT cells are present in a variety of tissues including liver, lung and blood against microorganism infections and cancers.

The invention disclosures a method of the identification of infections or tumors related to human T cell antigen receptors (TCRs) restricted to the monomorphic MHC class I-related to protein (MR1). MAIT cells contain a subset of αβ T lymphocytes displayed by a semi-invariant T cell receptor α (TCRα) chain. MAIT cells are also called MR1-restricted (MR1-R) T cells.

In healthy cells, MR1 is sparsely displayed on the cell surface but it is upregulated on the surface after cells are infected. Once MR1 is present or upregulated on the surface, MR1 associated with its ligand binds to the appropriate MR1-R T cells.

The invention also disclosures a method of the generation of MR1 restricted (MR1-R) T cells against microorganism infections and cancers.

Persistency of MR1-R T cells is critical for their functions in vivo.

In some embodiments, the extended persistency of MR1-R (MR1-restricted T cells) or TCR restricted T cells can be achieved by co-expressing the IL-15/IL-15 anchor.

In some embodiments, the extended persistency of MR1-R (MR1-restricted T cells) or TCR restricted T cell can be achieved by co-expressing the IL-15/IL-15sushi.

In some embodiments, MR1-R (MR1-restricted T cells) or TCR restricted T cells co-expressing IL-15/IL-15sushi or IL-15/IL-15sushi anchor can be scaled up and used as an off-the-shelf product.

In some embodiments, MR1-R (MR1-restricted T cells) or TCR restricted T cells co-expressing IL-15/IL-15sushi and IL-15/IL-15sushi anchor can be scaled up and used as an off-the-shelf product.

In one embodiment, MR1-R or TCR restricted T cells co-expressing IL-15/IL-15 sushi or IL-15/IL-15sushi anchor are capable of continuing supportive cytokine signaling, which is critical to their survival post-infusion in a patient.

In further embodiment, the extension of MR1-R (MR1-restrictedT cells) or TCR restricted T cell survival can be achieved by co-expressing a cytokine selected from a group of IL-7, IL-15, IL-15/IL-15 anchor, IL-15/IL-15RA, IL-12, IL-18 and IL-21.

In some embodiments, a MR1-R T cell (MR1-restricted T cells) or TCR restricted T cell expresses at least either a cytokine(s) and/or chemokine(s). Co-expressing cytokines in a MR1-R T cell or TCR restricted T cell can be selected from a group of cytokines including, but not limited to: IL-15/IL-15sushi, IL-15/IL-15sush anchor, IL-2, IL-4, IL-7, IL-10, IL-12, IL-18, IL-21, GM-CSF, and TGF-β. Co-expressing chemokines in a MR1-R T cell or TCR restricted T cell can also be selected from a group of chemokines including, but limited to: CCL2, CCL3, CCL4, CCL5, CCL7, CCL8, CCL19, CXCL1, CXCL2, CXCL9, CXCL10, or CXCL12 or CCL-21.

In some embodiments, a MR1-R T cell or TCR restricted T cell co-expresses IL-15/IL-15 anchor with at least one cytokine selected from a group of cytokines including, but not limited to, IL-15, IL-15/IL-15sushi, IL-2, IL-4, IL-7, IL-10, IL-12, IL-18, IL-21, GM-CSF, and TGF-β.

In some embodiments, a MR1-R T cell or TCR restricted T cell co-expresses IL-15/IL-15 anchor with IL-15.

In some embodiments, a MR1-R T cell or TCR restricted T cell co-expresses IL-15/IL-15 anchor with IL-15/IL-15sushi (FIGS. 2 and 11).

In some embodiments, a MR1-R T cell or TCR restricted T cell co-expresses IL-15/IL-15 anchor with IL-15 (11).

In some embodiments, a MR1-R T cell or TCR restricted T cell co-express IL-15/IL-15 anchor with IL-7(FIG. 10).

### Methods of Generating Engineered Cells

Any of the polynucleotides disclosed herein may be introduced into an engineered cell by any method known in the art.

In some embodiments of the present invention, any of the engineered cells disclosed herein may be constructed in a transposon system (also called a “Sleeping Beauty”), which integrates the gene or DNA into the host genome without a viral vector.

In one embodiment, to achieve enhanced safety profile or therapeutic index, the any of the engineered cells disclosed herein be constructed as a transient DAN or RNA-modified “biodegradable” version or derivatives, or a combination thereof. The RNA- or DNA modified versions of the present invention may be electroporated into T cells or NK cells.

Steps of methods of isolation of a MR1-R T cell capable of binding specifically to an antigen of infectious microorganism presented by a cell in associate with MR1antigen-presenting molecule:
a. Isolation of T cells from a patient or a health donor or umbilical cord blood
b. isolation an MR1-R T cell clone specifically reacts with a MR1 expressing cell infected with a viral particle or microorganism.
c. expansion of an MR1-R T cell clone

Steps of methods of isolation of a MR1-R T cell capable of binding specifically to an antigen presented by a cancer cell in associate with MR1antigen-presenting molecule:
a. Isolation of T cells from a patient or a health donor or umbilical cord blood
b. isolation an MR1-R T cell clone specifically reacts with a MR1 expressing cancer cells
c. expansion of an MR1-R T cell clone

In some embodiments, the isolation of MR1-R T clone comprises a step with the use of MACS (magnetic separation) or FACS (flow cytometry analysis) with markers of CD3⁺CD4⁻TCRγ/δ⁻ CD161^{high} interleukin-18 receptor^{high} or makers selected from a group, but limited to, of CD3, CD69, CD137 and CD150.

In some embodiments, invention disclosure provides a method of a MR1-R T cell T cell co-expressing secreted IL-15/IL-15sushi or IL-15/IL-15sushi anchor to enhance its expansion in vivo.

### Vectors

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the patient either in vivo or ex vivo. A number of retroviral systems are known in the art. In some embodiments, adenovirus vectors are used. A number of adenovirus vectors are known in the art. In one embodiment, lentivirus vectors are used.

Viral vector technology is well known in the art and is described, for example, in Sambrook et al, (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient and unique restriction endonuclease sites, and one or more selectable markers, (e.g., WO 01/96584; WO 01/29058; and U.S. Pat. No. 6,326,193).

Lentiviral vectors have been well known for their capability of transferring genes into human NK cells with high efficiency, but expression of the vector-encoded genes is dependent on the internal promoter that drives their expression. There are a wide range of promoters with different strength and cell-type specificity. Gene therapies rely on the ability of cells to express an adequate level of a protein and maintain expression over a long period of time. The EF-1α promoter has been commonly selected for the gene expression.

The present invention provides an expression vector containing a strong promoter for high level gene expression in NK cells or T cells. In further embodiment, the inventor discloses a strong promoter useful for high level expression of a gene in NK cells or T cells. In particular embodiments, a strong promoter relates to the SFFV promoter, which is selectively introduced in an expression vector to obtain high levels of expression and maintain expression over a long period of time in NK cells or T cells. Expressed genes prefer a cytokine or chemokine and NK or T cell co-stimulatory factors used for immunotherapy.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1 a (EF-1 a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the disclosure should not be limited to the use of constitutive promoters, inducible promoters are also contemplated as part of the disclosure. The use of an inducible promoter provides a molecular switch capable of turning on expression of the polynucleotide sequence, which is operatively linked when such expression is desired, or turning off the expression when expression is not desired. Examples of inducible promoters include, but are not limited to a metalothionine promoter, a glucocorticoid promoter, a progesterone promoter, and a tetracycline promoter.

Expression of chimeric antigen receptor polynucleotide may be achieved using, for example, expression vectors including, but not limited to, at least one of a SFFV (spleen-focus forming virus) or human elongation factor 11α (EF) promoter, CAG (chicken beta-actin promoter with CMV enhancer) promoter human elongation factor 1α (EF) promoter. Examples of less-strong/lower-expressing promoters utilized may include, but is not limited to, the simian virus 40 (SV40) early promoter, cytomegalovirus (CMV) immediate-early promoter, Ubiquitin C (UBC) promoter, and the phosphoglycerate kinase 1 (PGK) promoter, or a part thereof. Inducible expression of chimeric antigen receptor may be achieved using, for example, a tetracycline responsive promoter, including, but not limited to, TRE3GV (Tet-response element, including all generations and preferably, the 3rd generation), inducible promoter (Clontech Laboratories, Mountain View, CA) or a part or a combination thereof.

In a preferred embodiment, the promoter is an SFFV promoter or a derivative thereof. It has been unexpectedly discovered that SFFV promoter provides stronger expression and greater persistence in the transduced cells in accordance with the present disclosure.

“Expression vector” refers to a vector including a recombinant polynucleotide comprising expression control sequences operatively linked to a nucleotide sequence to be expressed. An expression vector includes sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide. The expression vector may be a bicistronic or multicistronic expression vector. Bicistronic or multicistronic expression vectors may include (1) multiple promoters fused to each of the open reading frames; (2) insertion of splicing signals between genes; fusion of genes whose expressions are driven by a single promoter; (3) insertion of proteolytic cleavage sites between genes (self-cleavage peptide); and (iv) insertion of internal ribosomal entry sites (IRESs) between genes.

In one embodiment, the disclosure provides an engineered cell having at least one chimeric antigen receptor polypeptide or polynucleotide.

An “engineered cell” means any cell of any organism that is modified, transformed, or manipulated by addition or modification of a gene, a DNA or RNA sequence, or protein or polypeptide. Isolated cells, host cells, and genetically engineered cells of the present disclosure include isolated immune cells, such as NK cells and T cells that contain the DNA or RNA sequences encoding a cytokine or a chimeric antigen receptor or chimeric antigen receptor complex and express the chimeric receptor on the cell surface. Isolated host cells and engineered cells may be used, for example, for enhancing an NK cell activity or a T lymphocyte activity for treatment of infectious diseases or cancers.

### The Invention Provides a Method for Treatment and Prevention of Infectious Diseases

In some embodiments, engineered immune cells are administered to a subject to prevent or inhibit infectious diseases. Infectious diseases include diseases associated with viral, fungal and bacterial infections. Viral infections include, but not limited to, coronaviruses (CoV), middle east respiratory syndrome (MERS-CoV), severe acute respiratory syndrome (SARS-CoV), China Wuhan Coronavirus (COVID-19), human T-cell lymphotrophic virus (HTLV) type I and II, immunodeficiency virus (HIV), cytomegalovirus, papilloma virus, polyoma virus, rabies virus, Sendai virus, poliomyelitis virus, coxsackievirus, rhinovirus, reovirus, rubella virus, adenovirus, Epstein-Barr virus and poxyvirus. Bacterial infections include, but not limited to, Streptococcus pyogenes, Streptococcus pneumoniae, Neisseria gonorrhoea, Neisseria meningitidis, Conynebacterium diphtheriae, Clostridium botulinum, Clostridium perfringens, Clostridium tetani, Haemophilus influenzae, Klebsiella pneumoniae, Klebsiella ozaenae, Klebsiella rhinoscleromoti, Staphylococcus aureus, Vibrio cholerae, Escherichia coli, Pseudomonas aeruginosa, Campylobacter (Vibrio) fetus, Campylobacter jejuni, Aeromonas hydrophila, Bacillus cereus, Edwardsiella tarda, Yersinia enterocolitica, Yersinia pest is, Yersinia pseudotiiberculosis, Shigella dysenteriae, Shigella sonnei, Salmonella typhimuhum, Treponema pallidum, Treponema pertenue, Treponema carateneum, Borrelia vincentii, Borrelia burgdorferi, Leptospira icier ohemorrhagiae, Mycobacterium tuberculosis, Toxoplasma gondii, Pneumocystis carinii, Francisella tularensis, Brucella abortus, Brucella suis, Brucella melitensis, Mycoplasma spp., Rickettsia prowazeki, Rickettsia tsutsugumushi, Chlamydia and Helicobacter pylori.

In some embodiments, engineered immune cells are administered to a subject to prevent or inhibit infectious diseases with agents including viral, fungal and bacteria.

The invention provides a method of treatment for proliferation disorders or cancers.

In some embodiments, engineered immune cells are administrated to a subject to treatment or inhibit neoplasms or cancers. The neoplasms or cancers include, but not limited to, leukemias, lymphoma, multiple myeloma, myeloid leukemia, chronic myeloproliferative neoplasms, myelodysplastic syndromes, chronic myeloid leukemia, sarcomas, colon carcinoma, lung cancer, brain cancer, pancreatic cancer, breast cancer, ovarian cancer, prostate cancer, squamous cell carcinoma, adenocarcinoma, medullary carcinoma, renal cell carcinoma, neuroendocrine tumors, and melanoma, metastases, or any disease characterized by uncontrolled cell growth or proliferation.

According to the disclosure, immune cells are T cells, NK T cells, macrophage, gamma delta T cells, NK cells, NK-92 cells, dendritic cells, MR-R T cells, CD4 cells and CD8 cells. In further embodiments, immune cells are derived from human peripheral blood mononuclear cells (PBMC), leukapheresis products (PBSC), human embryonic stem cells (hESCs), induced pluripotent stem cells (iPSCs), bone marrow, or umbilical cord blood.

As used herein, the terms “peptide,” “polypeptide,” and “protein” are used interchangeably, and refer to a compound having amino acid residues covalently linked by peptide bonds. A protein or peptide must contain at least two amino acids, and no limitation is placed on the maximum number of amino acids that can be included in a protein's or peptide's sequence. Polypeptides include any peptide or protein having two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, and fusion proteins, among others. The polypeptides include natural peptides, recombinant peptides, synthetic peptides, or a combination thereof.

A “signal peptide” includes a peptide sequence that directs the transport and localization of the peptide and any attached polypeptide within a cell, e.g. to a certain cell organelle (such as the endoplasmic reticulum) and/or the cell surface. As used herein, “signal peptide” and “leader sequence” are used interchangeably.

The signal peptide is a peptide of any secreted or transmembrane protein that directs the transport of the polypeptide of the disclosure to the cell membrane and cell surface, and provides correct localization of the polypeptide of the present disclosure. In particular, the signal peptide of the present disclosure directs the polypeptide of the present disclosure to the cellular membrane, wherein the extracellular portion of the polypeptide is displayed on the cell surface, the transmembrane portion spans the plasma membrane, and the active domain is in the cytoplasmic portion, or interior of the cell.

In one embodiment, the signal peptide is cleaved after passage through the endoplasmic reticulum (ER), i.e. is a cleavable signal peptide. In an embodiment, the signal peptide is human protein of type I, II, III, or IV. In an embodiment, the signal peptide includes an immunoglobulin heavy chain signal peptide.

### Combination Therapy

The compositions and methods of this disclosure can be used to generate a population of T lymphocyte or NK cells that deliver both primary and co-stimulatory signals for use in immunotherapy in the treatment of cancer and infection. In further embodiments, the present invention for clinical aspects are combined with other agents effective in the treatment of hyperproliferative diseases, such as anti-cancer agents. Anti-cancer agents are capable of reduction of tumor burdens in a subject. Anti-cancer agents include chemotherapy, radiotherapy and immunotherapy. In further embodiments, the present invention for clinical aspects are combined with other agents effective in the treatment of infection diseases, such as antibiotics agents, and so forth.

More than 50% of persons with cancer will undergo surgery of some type. Curative surgery includes resection in which all or part of cancerous tissue is physically removed, excised, and/or destroyed.

The compositions and methods described in the present disclosure may be utilized in conjunction with other types of therapy for cancer, such as chemotherapy, surgery, radiation, gene therapy, and so forth.

Further, NK cells are known to mediate anti-cancer effects without the risk of inducing graft-versus-host disease (GvHD).

The present disclosure may be better understood with reference to the examples, set forth below. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure

Administration of any of the engineered cells described herein may be supplemented with the co-administration of an enhancing agent. Examples of enhancing agents include immunomodulatory drugs that enhance immune cell activities, such as, but not limited to agents that target immune-checkpoint pathways, inhibitors of colony stimulating factor-1 receptor (CSF1R) for better therapeutic outcomes. Agents that target immune-checkpoint pathways include small molecules, proteins, or antibodies that bind inhibitory immune receptors CTLA-4, PD-1, and PD-L1, and result in CTLA-4 and PD-1/PD-L1 blockades. As used herein, enhancing agent includes enhancer as described above.

Administration of any of the engineered cells described herein may be supplemented with the co-administration of an enhancing agent. Examples of engineered cell enhancing agents can be selected from the group of an anti-CD40 antibody or CD40 ligand, an anti-OX 40 antibody, an anti-4-1BB antibody, a TNFR2-blocking antibody, an anti-CTLA4 antibody, a PD-L1 inhibitor, and a CpG oligonucleotide (CpG ODNs, TLR9 agonists).

In accordance with the present disclosure, an engineered cell can be used to express a CAR (chimeric antigen receptor) on its surface involving the treatment of a disease.

In accordance with the present disclosure, an engineered cell can be used to express T-cell receptors (TCRs)on its surface involving the treatment of a disease. TCR-engineered T (TCR-T) cells have promises against tumors and infection agent.

On this basis, the present disclosure also provides a method of providing long-term durable remission in patients by administering an engineered cell having a TCR polypeptide disclosed herein and co-expression of IL-15/IL-15sushi or IL-15/IL-15sushi anchor to increase the sensitivity of TCR recognition of target cancer cells or recruit innate immune cells to cancer cells or enhance TCR T cell persistency.

On this basis, the present disclosure also provides a method of providing long-term durable remission in patients by administering an engineered cell having a CAR polypeptide disclosed herein and co-expression of IL-15/IL-15sushi or IL-15/IL-15sushi anchor to increase the sensitivity of CAR recognition of target cancer cells or recruit innate immune cells to cancer cells or enhance CAR persistency.

Antigen-directed CAR immunotherapy, such as, but not limited to, CD19, CD20, CD22, CD2, CD3, CD4, CD5, CD7, CD52, CD38, CD33, CD30, CD123, GD2, CD45, CLL-1, BCMA, CS1, BAFF, TACI, and APRIL CAR.

In one embodiment, the target of the antigen recognition domain for CARs is selected from the group of, but not limited to, GD2, GD3, interleukin 6 receptor, ROR1, PSMA, PSCA (prostate stem cell antigen), MAGE A3, Glycolipid, glypican 3, F77, GD-2, WT1, CEA, HER-2/neu, DLL3, EGFR, folate receptor-alpha, EpCAM, CD171, CD117, mesothelin, GM2, DR5, EGFR, EpCAM, EpHA2, ER-alfa, gp100, LMP1, IL-13R, VEGFR-2, PSMA, PSCA, PD-L, MAGE-3, MAGE-4, MAGE-5, MAGE-6, alpha-fetoprotein, CA 19-9, CA 72-4, NY-ESO, FAP, ErbB, c-Met, MART-1, MUC1, MUC2, MUC3, MUC4, MUC5, MMG49 epitope, CD30, EGFRvIII, CLDN, CLDN18, CLDN18, CLDN18.2, CD33, CD123, CLL-1, NKG2D, NKG2D receptors, immunoglobin kappa and lambda, CD38, CD52, CD47, CD200, CD70, CD19, CD20, CD22, CD38, BCMA, CS1, BAFF receptor, TACI, CD3, CD4, CD8, CD5, CD7, CD2, and CD138.

In one embodiment, the engineered cell with CD19-15S-15RA-Q-TM includes a CD19 chimeric antigen receptor polypeptide, secreting IL-15 and IL-15/IL-15sushi anchor ((SEQ ID NO. 7), and corresponding nucleotides (SEQ ID NO. 8).

In one embodiment, the engineered cell with CD19-15RA-Q-TM includes a CD19 chimeric antigen receptor polypeptide, secreting IL-15/IL-15sushi and IL-15/IL-15sushi anchor ((SEQ ID NO. 9), and corresponding nucleotides (SEQ ID NO. 10).

In one embodiment, the engineered cell with CD19-RQR-7xp-TM includes a CD19 chimeric antigen receptor polypeptide, secreting IL-7 and IL-15/IL-15sushi anchor ((SEQ ID NO. 11), and corresponding nucleotides (SEQ ID NO. 12).

In one embodiment, the engineered cell with CD19-RQR-7xp includes a CD19 chimeric antigen receptor polypeptide and secreting IL-7 ((SEQ ID NO. 13), and corresponding nucleotides (SEQ ID NO. 14).

In one embodiment, the engineered cell with CD19-RQR-TM includes a CD19 chimeric antigen receptor polypeptide and IL-15/IL-15sushi anchor ((SEQ ID NO. 15), and corresponding nucleotides (SEQ ID NO. 16).

As used herein, “patient” includes mammals. The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). Preferably, the mammal is a human. A patient includes subject.

In certain embodiments, the patient is a human 0 to 6 months old, 6 to 12 months old, 1 to 5 years old, 5 to 10 years old, 5 to 12 years old, 10 to 15 years old, 15 to 20 years old, 13 to 19 years old, 20 to 25 years old, 25 to 30 years old, 20 to 65 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old.

The terms “effective amount” and “therapeutically effective amount” of an engineered cell as used herein mean a sufficient amount of the engineered cell to provide the desired therapeutic or physiological or effect or outcome. Such, an effect or outcome includes reduction or amelioration of the symptoms of cellular disease. Undesirable effects, e.g. side effects, are sometimes manifested along with the desired therapeutic effect; hence, a practitioner balances the potential benefits against the potential risks in determining what an appropriate “effective amount” is. The exact amount required will vary from patient to patient, depending on the species, age and general condition of the patient, mode of administration and the like. Thus, it may not be possible to specify an exact “effective amount”. However, an appropriate “effective amount” in any individual case may be determined by one of ordinary skill in the art using only routine experimentation. Generally, the engineered cell or engineered cells is/are given in an amount and under conditions sufficient to reduce proliferation of target cells.

Following administration of the delivery system for treating, inhibiting, or preventing a cancer, the efficacy of the therapeutic engineered cell can be assessed in various ways well known to the skilled practitioner. For instance, one of ordinary skill in the art will understand that a therapeutic engineered cell delivered in conjunction with the chemo-adjuvant is efficacious in treating or inhibiting a cancer in a patient by observing that the therapeutic engineered cell reduces the cancer cell load or prevents a further increase in cancer cell load. Cancer cell loads can be measured by methods that are known in the art, for example, using polymerase chain reaction assays to detect the presence of certain cancer cell nucleic acids or identification of certain cancer cell markers in the blood using, for example, an antibody assay to detect the presence of the markers in a sample (e.g., but not limited to, blood) from a subject or patient, or by measuring the level of circulating cancer cell antibody levels in the patient.

Throughout this specification, quantities are defined by ranges, and by lower and upper boundaries of ranges. Each lower boundary can be combined with each upper boundary to define a range. The lower and upper boundaries should each be taken as a separate element.

Reference throughout this specification to “one embodiment,” “an embodiment,” “one example,” or “an example” means that a particular feature, structure or characteristic described in connection with the embodiment or example is included in at least one embodiment of the present embodiments. Thus, appearances of the phrases “in one embodiment,” “in an embodiment,” “one example,” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

As used herein, the terms “comprises,” “comprising,” “includes,” “including,” “has,” “having,” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus.

Further, unless expressly stated to the contrary, “or” refers to an inclusive “or” and not to an exclusive “or”. For example, a condition A or B is satisfied by any one of the following: A is true (or present) and B is false (or not present), A is false (or not present) and B is true (or present), and both A and B are true (or present).

Additionally, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.”

In this specification, groups of various parameters containing multiple members are described. Within a group of parameters, each member may be combined with any one or more of the other members to make additional sub-groups. For example, if the members of a group are a, b, c, d, and e, additional sub-groups specifically contemplated include any one, two, three, or four of the members, e.g., a and c; a, d, and e; b, c, d, and e; etc.

As used herein, a XXXX antigen recognition domain is a polypeptide that is selective for XXXX. “XXXX” denotes the target as discussed herein and above. For example, a CD19 antigen recognition domain is a polypeptide that is specific for CD19

As used herein, CDXCAR refers to a chimeric antigen receptor having a CDX antigen recognition domain.

### EXAMPLES

### Secreting IL-15/IL-15sushi

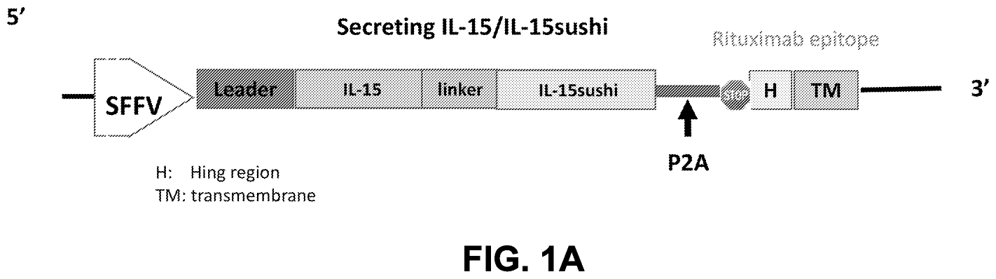

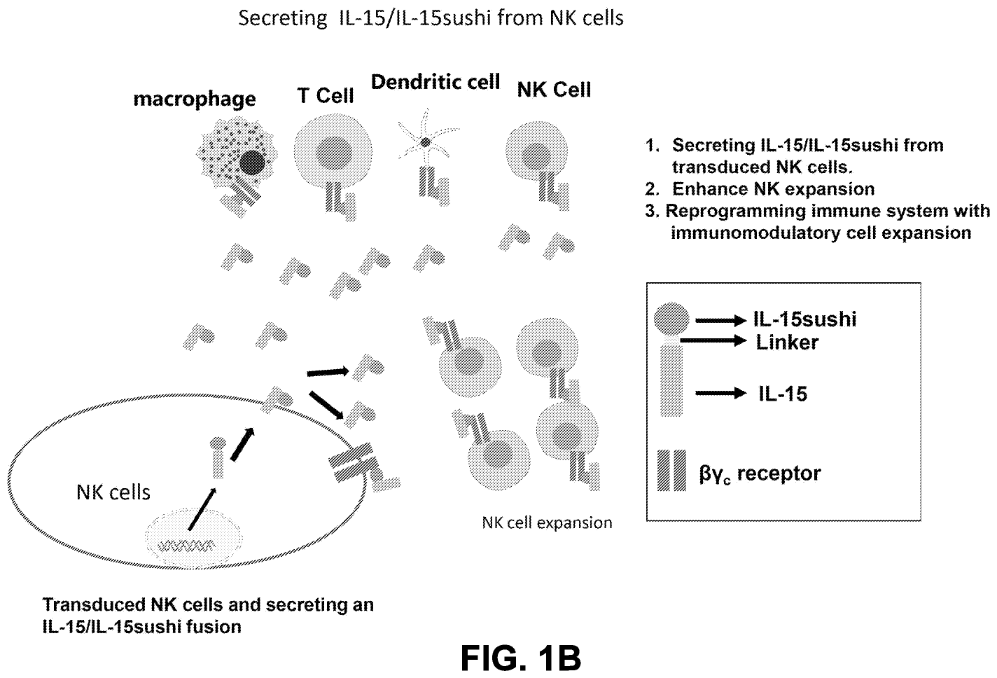

The structural organization of a secreting IL-15/IL-15sushi construct with a rituximab epitope is shown in FIG. 1A. Links by P2A schematic to generate a super1 CAR showing a CAR, GD2 CAR equipped with 4-1BBL and IL-15/IL-15sushi in a single construct. The construct consists of a SFFV promoter driving the expression of two segments in a single construct. The secreting IL-15/IL-15sushi fusion protein and rituximab safety switch in the construct splits after expression. Secreting IL-15/IL-15sushi (enhancer) comprises a leader sequence (IL-2) fused to IL-15/IL-15sushi. Rituximab safety protein comprises a leader sequence, two copies of rituximab epitopes, a hinge (H) region, a transmembrane domain (TM). Rituximab safety protein is anchored on the surface of a cell. The soluble IL-15/IL-15sushi fusion are stable and functions as an unexpected and powerful immunomodulatory for T/NK cells, dendritic cells, macrophages and their neighbor tumor immune response cells (FIG. 1B). The soluble IL-15/IL-15sushi fusion is also able to enhance T/NK cell persistency, stimulate T/NK cell functions of anti-pathogen or anti-tumor activities. The soluble IL-15/IL-15sushi fusion provides vaccine-like effects by reprogramming body's immune system to fight infections and cancers.

### IL-15/IL-15sushi Anchor on the Surface of a Cell

The construct with IL-15/IL-15sushi anchor is shown in FIG. 2A. An IL-15/IL-15sushi anchor construct consists of an SFFV promoter driving the expression of an IL-15/IL-15sushi anchor (also called anchor). The IL-15/IL-15sushi portion of anchor is composed of IL-2 signal peptide (or signal peptides from IL-15Rα receptor or influenza virus hemagglutinin, HA). IL-15 is fused to sushi domain of IL-15 alpha receptor via a 26-amino acid poly-proline linker. The anchor comprises two copies of rituximab biding sequence in a hinge (H) region, a transmembrane domain (TM). IL-15/IL-15sushi anchor provides a synergistic effect of NK or T cell activation or anti-infection or anti-tumor activity (FIG. 2B). IL-15/IL-15sushi is anchored on the surface of T or NK cells, which also enhances NK and T cell expansion and persistency.

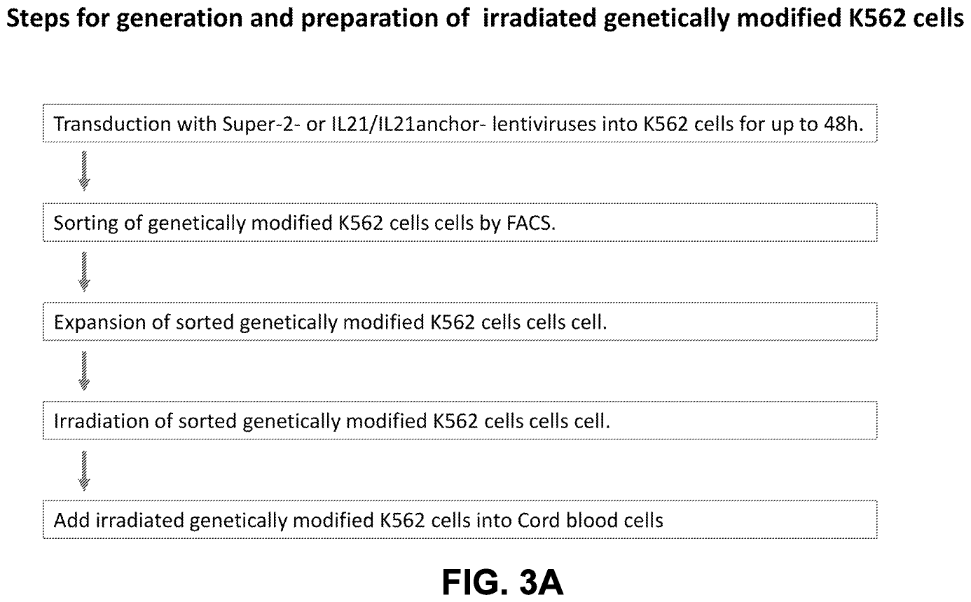

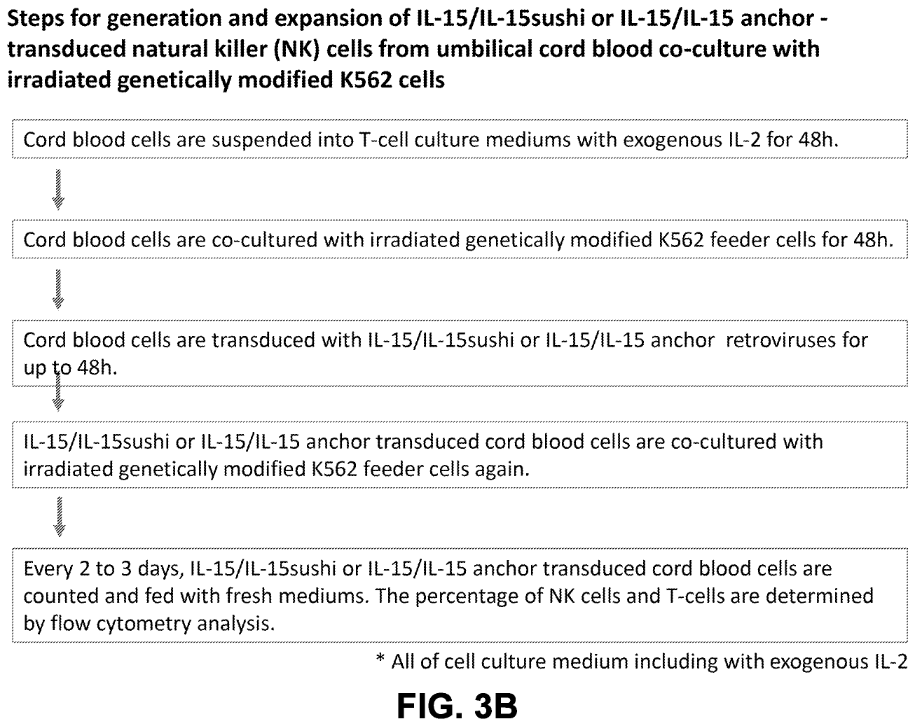

The IL-15 can be a variant, IL-15N72D described in elsewhere, U.S. Pat. No. 8,507,222 B2 Expansion of NK cells from human cord blood (key steps shown in FIGS. 3A and 3B)

### Generation of Feeder Cells

The steps for generation of feeder cells are shown in FIG. 3 with a flowchart. K562 cells are transduced with lentiviruses expressing a surface anchor protein or scFv tagged IL-21 (IL-21 anchor) (SEQ ID NO. 17 and 18) or scFv tagged 4-1BBL and IL-15/IL-15sushi anchor (also called super 2)(SEQ ID NO. 19 and 20).

In one embodiment, the engineered K562 cell includes IL-21 anchor polypeptide (SEQ ID NO. 17), and corresponding nucleotides (SEQ ID NO. 18).

In one embodiment, the engineered K562 cell includes super2 polypeptide (SEQ ID NO. 19), and corresponding nucleotides (SEQ ID NO. 20).

K562 were transduced with IL-21 anchor or super 2 lentiviruses for 48 hours. After transduction, cells are expanded and labeled by antibodies for sorting of genetically modified K562 cells by FACS. Sorted genetically modified K562 cells are expanded, irradiated (10-100 Gy) and frozen down until use. Irradiated genetically modified K562 cells are added into cord blood cell to stimulate and expand NK cells as feeder cells.

### Expansion of Human NK Cells from Human Cord Blood (FIG. 3B)

Flowchart (FIG. 3B) shows the steps for generation and expansion of transduced natural killer (NK) cells from umbilical cord blood by co-culture with irradiated genetically modified K562 cells. Cord blood cells are suspended in T-cell culture mediums with 300 U/ml IL-2 for 48 h. Irradiated genetically modified K562 cells are added into cord blood cells to stimulate and expand NK cells for 48 h.

Cord blood cells are stimulated for up to 48 h. The cord blood cells are co-cultured with irradiated genetically modified K562 feeder cells again.

Every 2 to 3 days, the cord blood cells are counted and fed with fresh mediums to maintain cell condition. Exogenous IL-2 is added to all of cell culture media.

After 2 weeks, expansion of NK cells increases 220-680-fold compared to first day. After 3 weeks, fold expansion of NK cells becomes 450-1500 times compared to first day.

The percentage of NK cells and T-cells are determined by flow cytometry analysis using antibodies against human CD3 and CD56

### Evaluation of Persistence of Infused Secreting IL-15/IL-15sushi Transduced NK Cells in Vivo

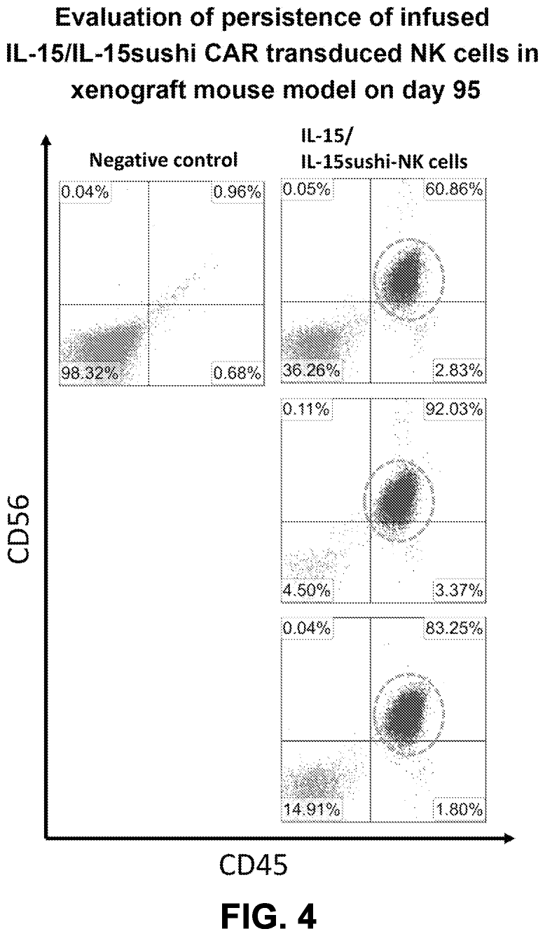

In order to evaluate the persistence of IL-15/IL-15sushi NK cells, we developed a xenogeneic mouse model using NSG mice sub-lethally irradiated and intravenously injected with 1×10⁶ of luciferase-expressing MM.1S multiple myeloma cells to induce measurable tumor formation. On Day 4, leukemic mice were intravenously injected with 10×10⁶ IL-15/IL-15sushi NK cells derived human cord blood. Evaluation of persistence of infused IL-15/IL-15sushi transduced NK cells in xenograft mouse model were done on Day 95 (FIG. 4). The peripheral blood was collected from individual mice and cells were labeled using anti-human CD56-and CD45 antibodies to detect the presence of infused control- and/or IL-15/IL-15sushi-transduced NK cells. Control NK cells were undetectable about a week post-infusion. It was surprisingly found that IL-15/IL-15sushi transduced NK cells persisted more than 90 days post-infusion (FIG. 4). In general, human non-transduced NK cells usually persist less than one or two weeks in mice.

The NK cell is an ideal platform against tumors or infections if NK cells can persist a relatively long period of time. However, the life expectancy of NK CAR cells in vivo is very short, with a lifespan of one or two weeks. Ideally, the NK cell persistency should be one or two months to be considered adequate for therapy. We have developed a NK cell platform for a universal therapy with improved persistency and killing using secreting IL-15/IL-15sushi fusion. The invented studies demonstrate NK cells co-expressing secretory IL-15/IL-15sushi can be used as a universal platform for treatment of a variety of diseases.

### Determination if IL-15 Being Secreted in Transduced NK Cells

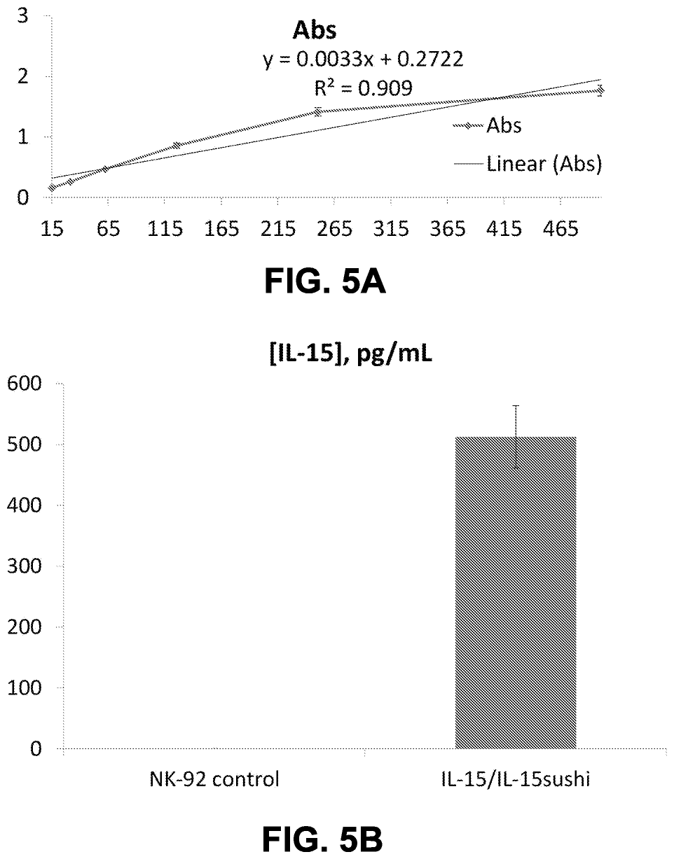

To determine if IL-15 is being secreted, NK-92 cell line was transduced with lentiviral vector expressing IL15/IL-15sushi. Cells were sorted on BD FACS Aria to select transduced NK cells. Sorted cells were expanded, and after 72 hours supernatant was collected and subjected to ELISA on 96-well plates precoated with IL-15 antibody. Following manufacturer's (Boster) directions, colorimetric results obtained on a plate reader were compared to a standard curve generated with human IL-15 to determine concentration of IL-15 in the supernatant (FIG. 5). It was determined that IL-15 was detected in the supernatant at ˜500 pg/mL. By comparison, supernatant containing approximately the same number of wild-type control NK-92 cells had a background concentration.

### Evaluation of Persistence of IL-7-IL-15/IL-15sushi Anchor in Vivo

In order to evaluate the persistence of IL-7-IL-15/IL-15sushi T cells, we developed a xenogeneic mouse model using NSG mice sub-lethally irradiated and intravenously injected with a very low dose, 1.6×10⁵ of IL-7-IL-15/IL-15sushi anchor transduced T cells. Peripheral blood was collected from individual mice and cells were labeled using anti-human CD56- and CD3 antibodies to detect the presence of infused control and transduced T cells. The persistence of control T cells or IL-7-IL-15/IL-15sushi anchor transduced T cells in collected peripheral blood was determined by flow cytometry analysis. After infusion, while control T cells were detected in mice at very low level, 0.2% 24 hours post-infusion and this population became undetectable 5 days post-infusion. In contrast, IL-7-IL-15/IL-15sushi anchor transduced T cells were expanded and detected starting day 5 post-infusion and reached a peak at day 42 days and gradually dropped at 49 days post-infusion.

### Generation of 4LV-Q-IL-15R Comprising an Immunoglobulin FAB Light Chain Tag, Rituximab Epitopes and Secreting IL-15/IL-15sushi

For generation of a high level of 4LV-Q-IL-15R expression, the Lenti-X 293T cell line was used as packaging cells to generate lentiviruses expressing 4LV-Q-IL-15R. Activated human peripheral blood T cells were transduced with the lentiviral vector or 4LV-Q-IL-15R.

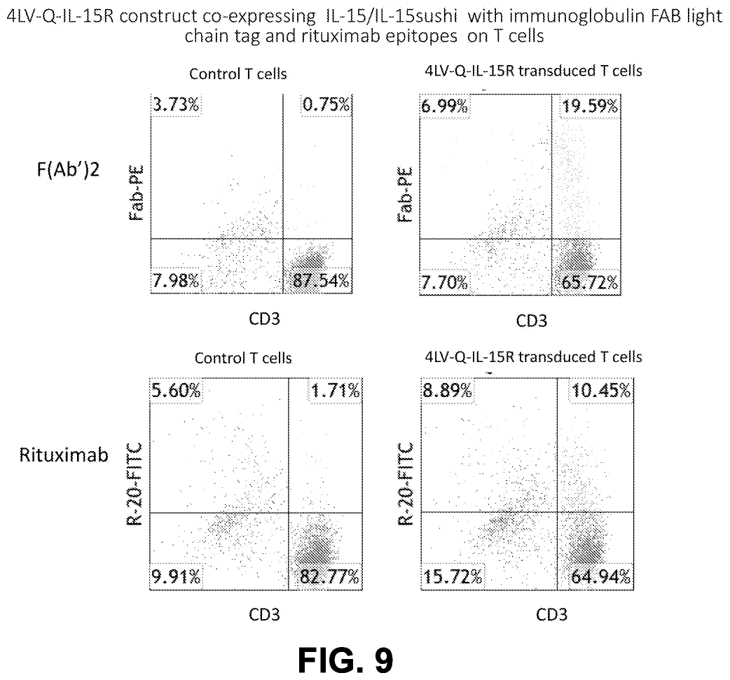

FIG. 8 shows a schematic of a 4LV-Q-IL-15R construct co-expressing secreting IL-15/IL-15sushi with immunoglobulin FAB light chain tag and rituximab epitopes (also called 4LV-Q-IL-15R). FIG. 9 shows the transduction efficiency between activated T cells transduced with either control lentiviruses or 4LV-Q-IL-15R lentiviruses, as determined by labeling with goat anti-mouse F(Ab′) 2 antibody or conjugated rituximab antibody. Activated T cells transduced with the 4LV-Q-IL-15R viruses resulted in 19.58% F(Ab′)2 positive cells and 4LV-Q-IL-15R transduced T cells were detected by a rituximab antibody.

### Generation of GL-Q-7xp-TM Construct Containing an IL-15/IL-15 Anchor with an Immunoglobulin FAB Light Chain Tag (GL), Rituximab Epitopes and a Secreting IL-7

IL-15 functions through a trimeric IL-15R complex, which contains a high affinity binding α-chain (IL-15 Rα) and the common IL-2R β- and γ-chains. IL-15 secreting from a cell binds to IL-15 Rα associated with IL-15 receptor β- and γ-chains on the surface of cells.

A 65 amino acid sequence of the extracellular portion of IL-15Rα, called sushi domain involves the binding of IL-15. It has been known that the cytoplasmic domain of IL-15 receptor a chain is critical for normal IL-15Rα functions.

Prior to the art, IL-15Rα (full-length of IL-15 receptor alpha subunit) accelerates leukemia development in T cells when constitutively co-expressed with IL-15 (Sato et al, Blood. 2011 Apr. 14; 117) in transgenic mouse models (Sato et al, Blood. 2011 Apr. 14; 117). To reduce the risk of tumor formation, only 65 amino acid segment of the extracellular portion of IL-15 sushi domain involving the binding of IL-15 was selected instead of the full length of IL-15Rα and fused to IL-15 and then expressed it on the surface of immune cells. However, this omission was compensated for by the incorporation into the design of either secreted IL-7 or IL-15 or IL-15/IL-15sushi. This secretion can be easily controlled using a safety switch (FIG. 10 and FIG. 11), thereby turning off expression in adverse conditions.

GL-Q-7xp-TM construct comprises a SFFV promoter driving the expression of a rituximab safety switch and secreting IL-7 and an IL-15/IL-15sushi anchor linked by P2A and T2A self-cleavage peptides, respectively. Upon cleavage of these P2A and T2A peptides, enhancers, rituximab safety switch protein and IL-7 and IL-15/IL-15sushi anchor are separated. Rituximab safety protein comprises a leader sequence, an immunoglobulin FAB light chain tag, two copies of rituximab epitopes, a hinge (H) region, a transmembrane domain (TM). Secreting IL-7 (enhancer) comprises a leader sequence and IL-7 protein. The IL-15/IL-15sushi anchor is composed of a signal peptide fused to IL-15 and linked to sushi domain of IL-15 alpha receptor via a 26-amino acid poly-proline linker, hinge (H) region and a transmembrane domain (TM). IL-15/IL-15sushi is anchored on the surface of T or NK cells, which results in enhancing NK and T cell expansion and persistency. Secreting IL-7 enhances IL-15/IL-15sushi anchor's functions in immune cell expansion and persistency.

An engineered cell with GL-Q-7xp-TM was in prepared in accordance with the present disclosure (FIG. 9). GL-Q-7xp-TM NK cells to lyse leukemia/lymphoma or cancer cells or infectious agents.

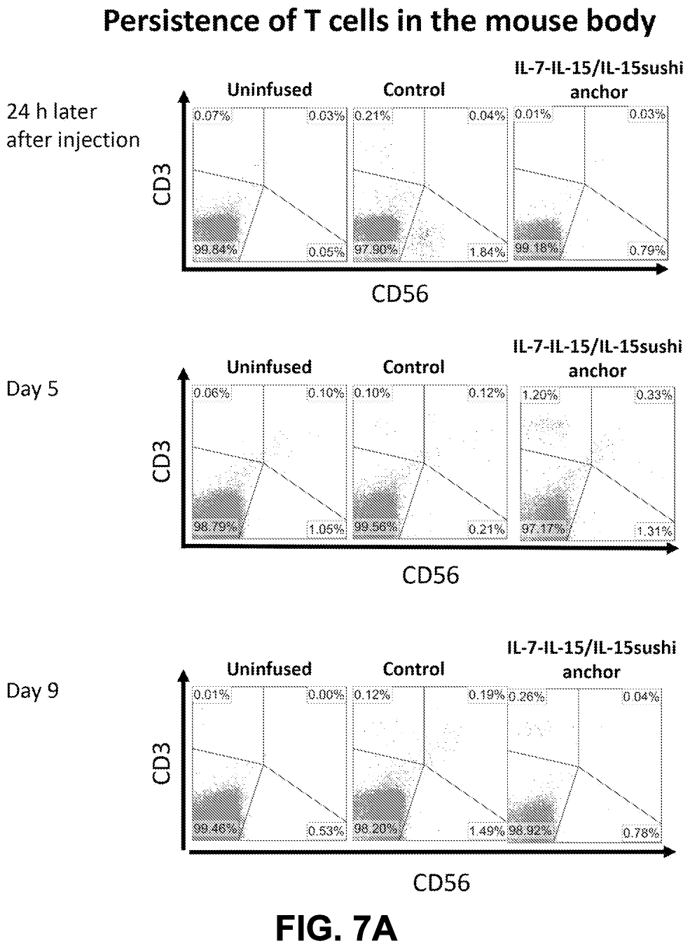

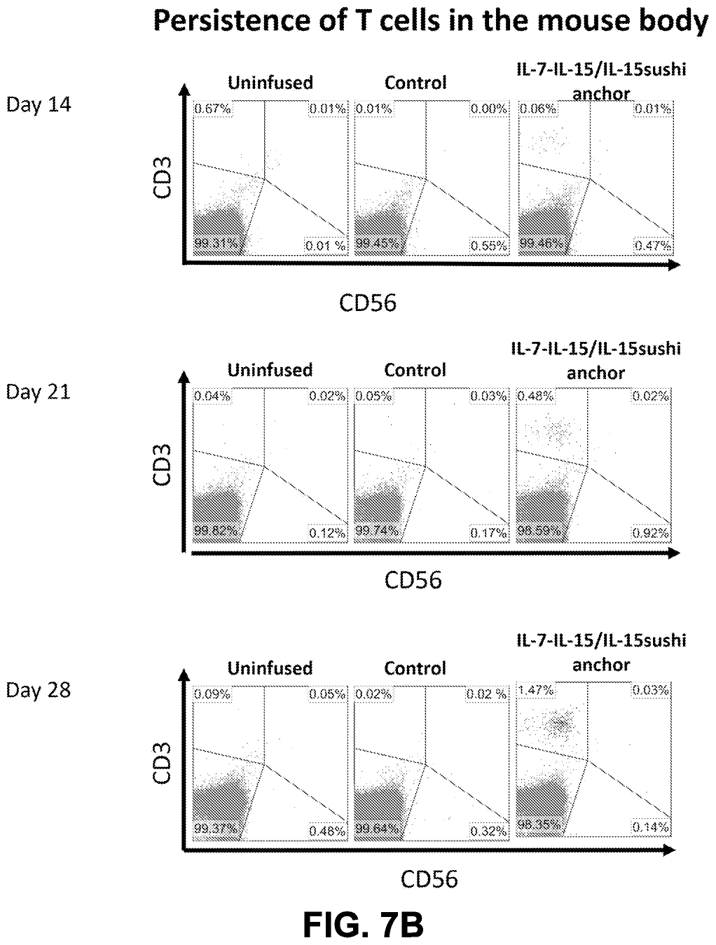

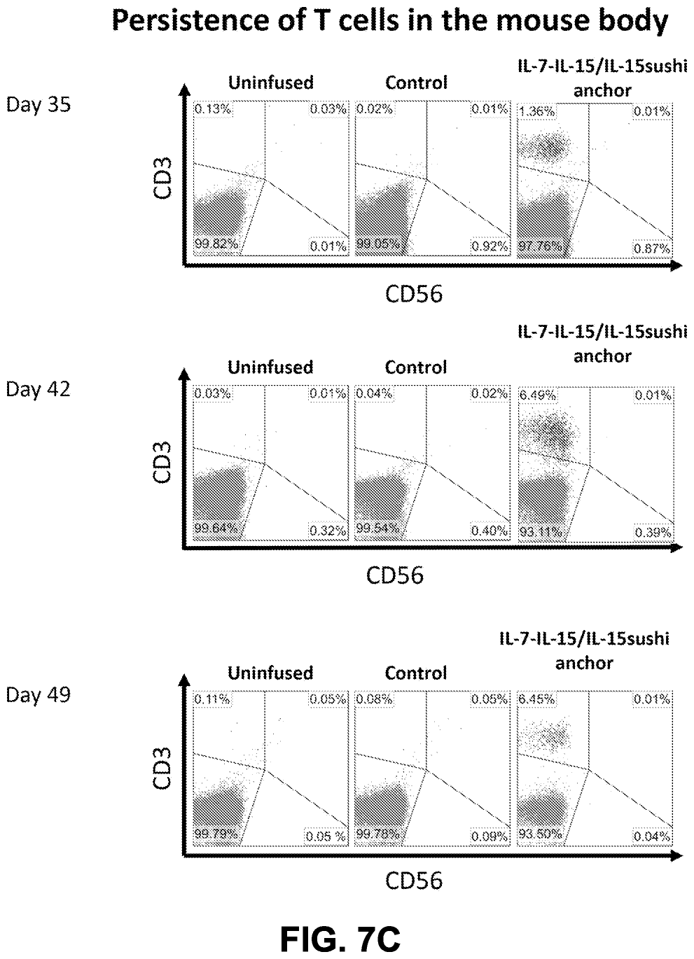

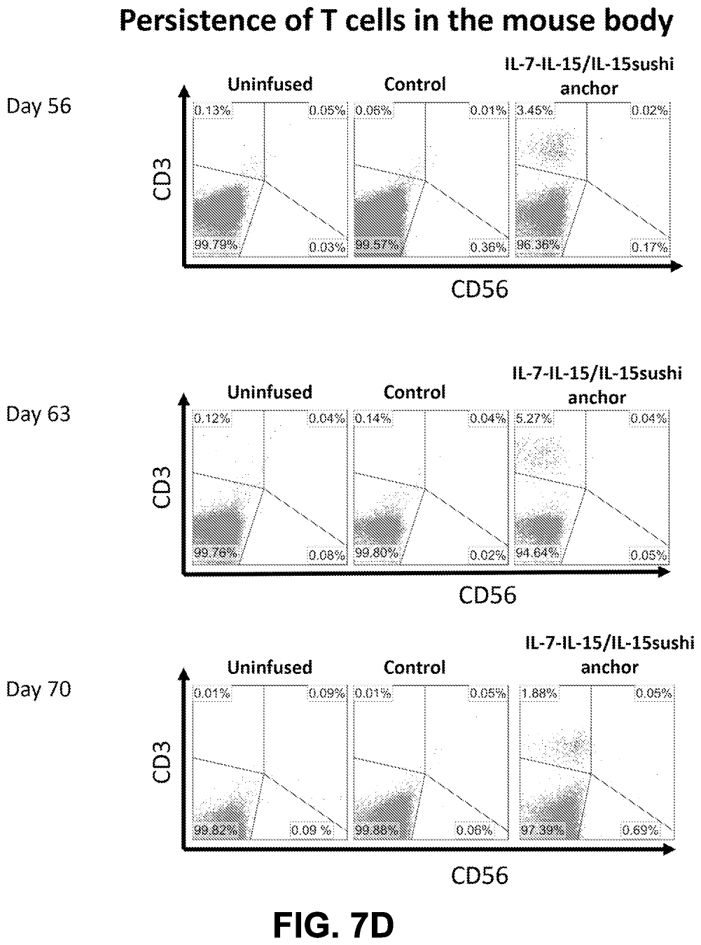

Similar assays for engineered cell persistency were in accordance with the present disclosure (FIG. 7).

In vivo anti-tumor activities, cell killing is performed in a xenogeneic mouse model using methods described in PCT/US2016/019953 and PCT/US2016/039306

### Generation of GL-Q-IL-15R-TM Construct Containing IL-15/IL-15 Anchor with Immunoglobulin FAB Light Chain Tag (GL), Rituximab Epitopes and Secreting IL-15/IL-15sushi

To reduce the risk of tumor formation, only 65 amino acid segment of the extracellular portion of IL-15 sushi domain involving the binding of IL-15 was selected instead of the full length of IL-15Rα and fused to IL-15 and then expressed it on the surface of immune cells. However, this omission was compensated for by the incorporation into the design of either secreted IL-15/IL-15sushi. This secretion can be easily controlled using a safety switch (FIG. 10 and FIG. 11), thereby turning off expression in adverse conditions.

The construct consists a SFFV promoter driving the expression of a rituximab safety switch and secreting IL-15/IL-15sushi and an IL-15/IL-15sushi anchor linked by P2A and T2A self-cleavage peptides, respectively. Upon cleavage of these P2A and T2A peptides, enhancers, rituximab safety switch protein and IL-15/IL-15sushi and IL-15/IL-15suhi anchor are separated. Rituximab safety protein comprises a leader sequence, an immunoglobulin FAB light chain tag, two copies of rituximab epitopes, a hinge (H) region, a transmembrane domain (TM). Secreting IL-15/IL-15sushi (enhancer) comprises a leader sequence and IL-IL-15/IL-15sushi protein. The IL-15/IL-15sushi anchor is composed of a signal peptide fused to IL-15 and linked to sushi domain of IL-15 alpha receptor via a 26-amino acid poly-proline linker, hinge (H) region and a transmembrane domain (TM). IL-15/IL-15sushi is anchored on the surface of T or NK cells, which results in enhancing NK and T cell expansion and persistency. Secreting IL-15/IL-15sushi enhances IL-15/IL-15sushi anchor's functions in immune cell expansion and persistency.

An engineered cell with GL-Q-IL-15/IL-15sushi-TM was prepared in accordance with the present disclosure (FIG. 9). GL-Q-IL-15/IL-15sushi-TM NK cells to lyse leukemia/lymphoma or cancer cells or infectious agents.

Similar assays for engineered cell persistency were in accordance with the present disclosure (FIG. 7).

In vivo anti-tumor activities, cell killing is performed in a xenogeneic mouse model using methods described in PCT/US2016/019953 and PCT/US2016/039306

In one embodiment, the engineered cell with 4LV-Q-IL-15R construct includes polypeptides of secreting IL-15/IL-15sushi, immunoglobulin FAB light chain tag and rituximab epitopes (SEQ ID NO. 1) and corresponding nucleotides (SEQ ID NO. 2).

In one embodiment, the engineered cell with GL-Q-7xp-TM construct has polypeptides of IL-15/IL-15sushi anchor, immunoglobulin FAB light chain tag, rituximab epitopes and secreting IL-7 (SEQ ID NO. 3) and corresponding nucleotides (SEQ ID NO. 4).

In one embodiment, the engineered cell with GL-Q-IL-15R-TM construct has IL-15/IL-15 anchor with immunoglobulin FAB light chain tag (GL), rituximab epitopes and secreting IL-15/IL-15sushi (SEQ ID NO. 5) and corresponding nucleotides (SEQ ID NO. 6).

### Examples of IL-15/IL-15sushi Enhances Immune Cell Functions

We disclosed the invention of immune cells, T cells expressing IL-15/IL-15sush with a CAR, CD19 CAR. This construct is called CD19b-IL-15/IL-15sushi which has a CD19 CAR and secreting IL-15/IL-15sushi. This system allows us to test how IL-15/IL-15sushi enhances immune cell functions.

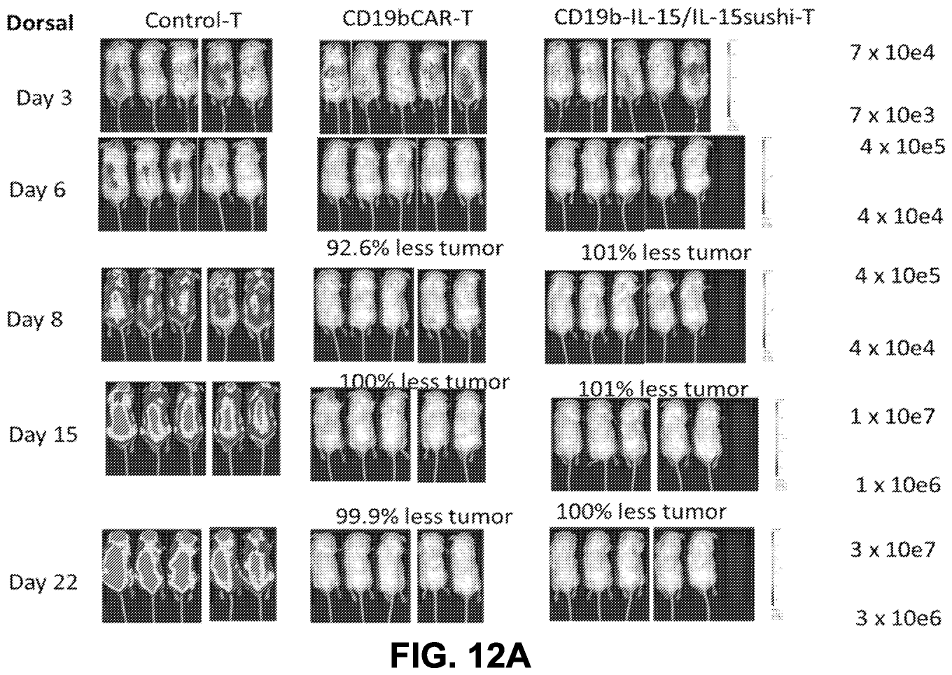

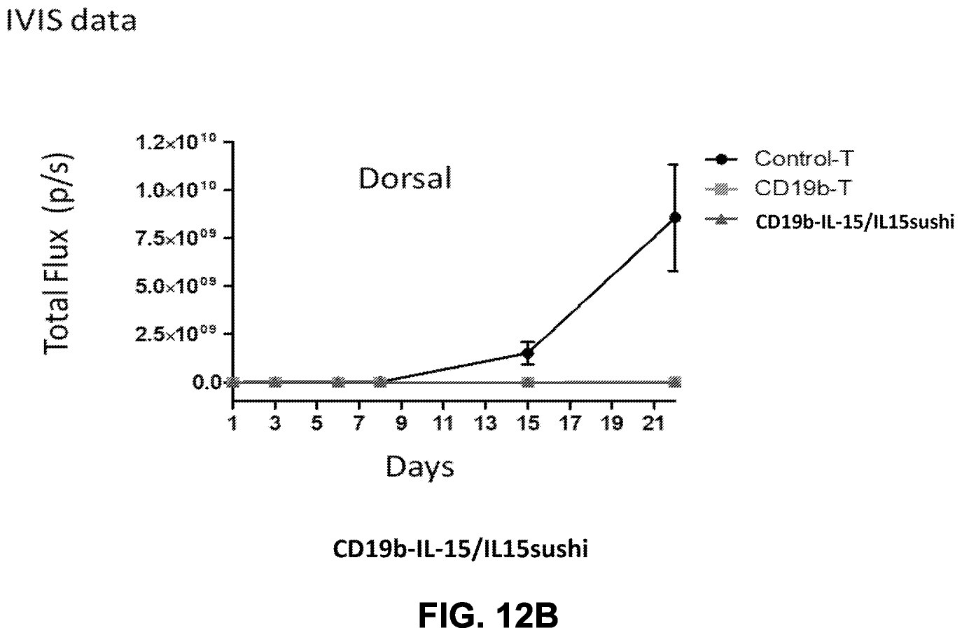

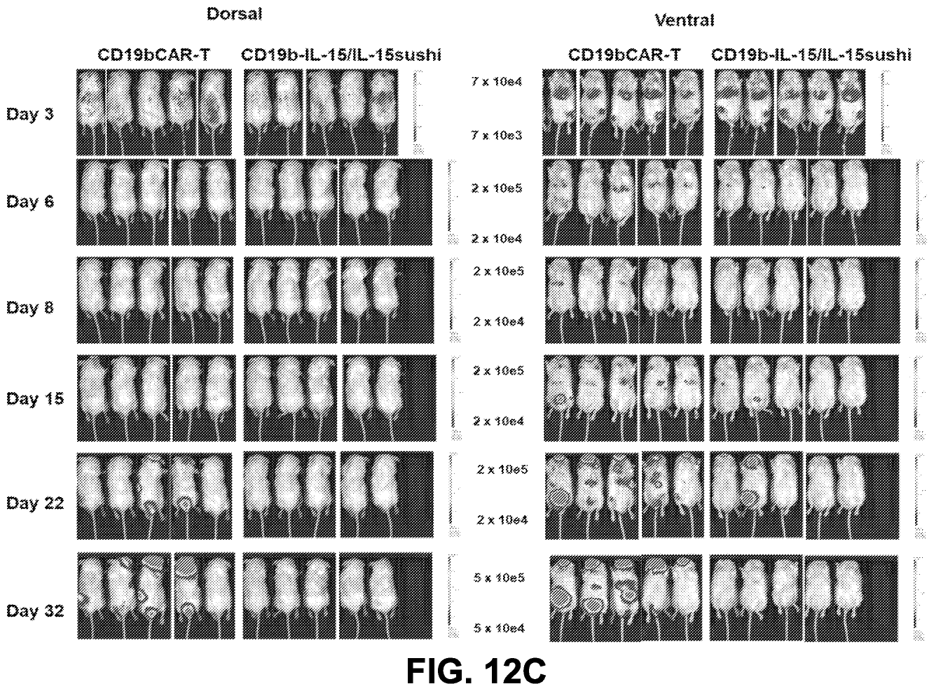

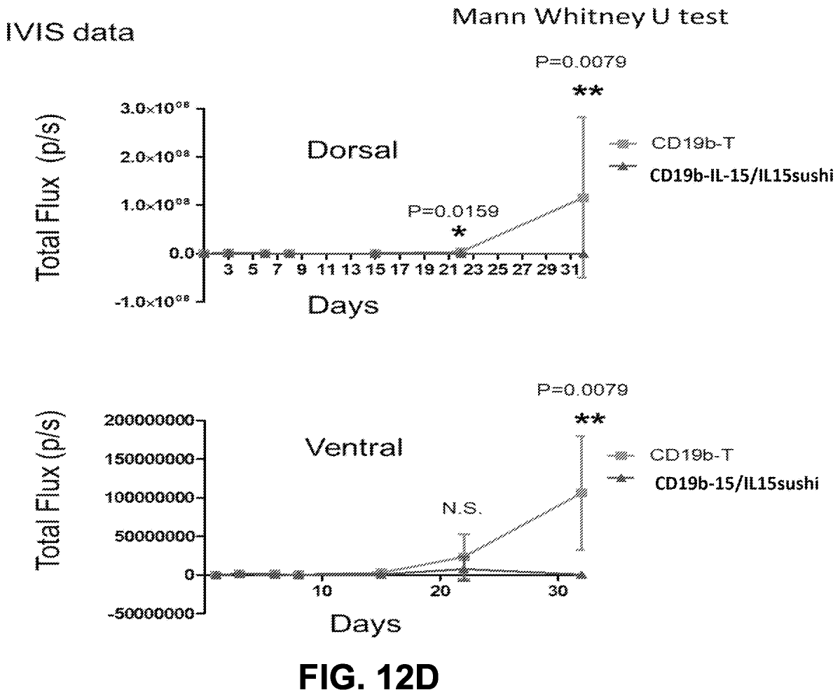

To test CD19b-IL-15/IL-15sushi CAR function in vivo, we established xenogeneic mouse models. Mice were injected with Reh tumor cells (0.5×10⁶ cells/mouse) expressing luciferase on Day 1 (FIG. 12A). On Day 3, IVIS was conducted to assay the appearance of circulating Reh cells. On Day 4, control T-cells, CD19b CAR, and CD19b-IL15/IL15sushi CAR T-cells were injected (˜7.5×10⁶ total cells/mouse) and on day 6 through 22, IVIS imaging was conducted to assay semi-quantitative assessment of tumor burden and subsequent tumor depletion and control of cell growth by T-cells. Here, both CAR T treatments demonstrated similar efficacy, with the IL-15 armored CAR demonstrating comparable or better control of the Reh tumor growth when compared to standard CART19 cells. It was found that CD19 based CARs deplete Reh cells in vivo and IL15/IL15sushi conjugates augment anti-tumor response. A line graph was then constructed, plotting IVIS values (estimation of tumor burden) against time for the treatment cohorts (FIG. 12B). As the tumor burden rises within the control group, both CAR T groups show steady maintenance of tumor suppression with significantly decreased tumor counts as measured by statistical analysis.

We then performed a long-term comparison CD19b-CAR-T vs CD19b-IL-15/IL15sushi CAR-T against REH cells using a similar experimental scheme with identical IVIS methodology as described in FIG. 12A; however, mice were followed until signs of tumor relapse were seen (FIG. 12C). Here, after day 30, we observed that aggressive Reh tumor relapse began to occur in standard CART19 treated mice. Clusters of tumor (indicated by red regions on the IVIS imaged mice) are seen in most CART19 mice, with a single CD19b-IL-15/IL-15sushi CART treated mice also showing tumor growth by day 22. However, after day 30, all CART19 mice showed signs of severe tumor relapse, while CD19b-IL-15/IL-15sushi CAR T treated mice showed no sign of tumor. Even the relapsed mouse on day 22 was absolved of its tumor by day 32, signifying that CD19b-IL-15/IL-15sushi CAR T cells were still in effective circulation.

A line graph was then created to summarize IVIS trend values estimating tumor growth over time for each treatment cohort (FIG. 12D). Past day 30, the tumor burden for the standard CD19b CAR (CART19) treated mice rises precipitously resulting in highly significant increases in tumor burden compared to the CD19b-IL-15/IL-15sushi armored CAR T treatment group which remained largely tumor free. Values are displayed for both views of the mice (ventral and dorsal image acquisition views). As time passed, Rch tumor relapsed in standard CAR T treatment; however, the armored CAR persisted and depleted relapsed tumor, keeping mice disease free.

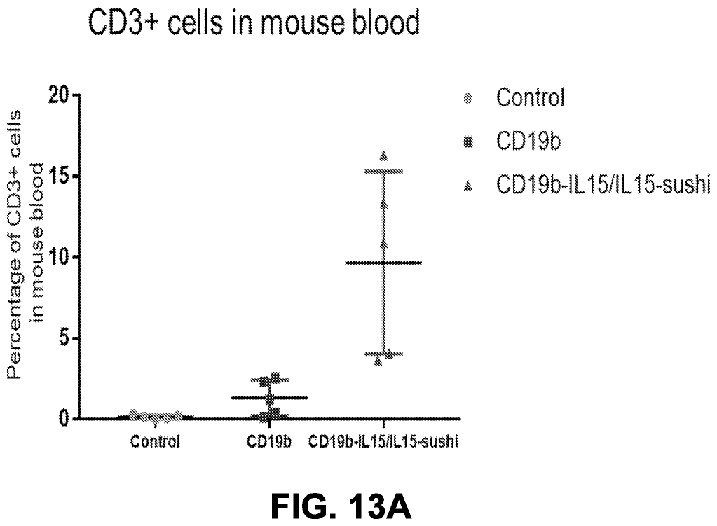

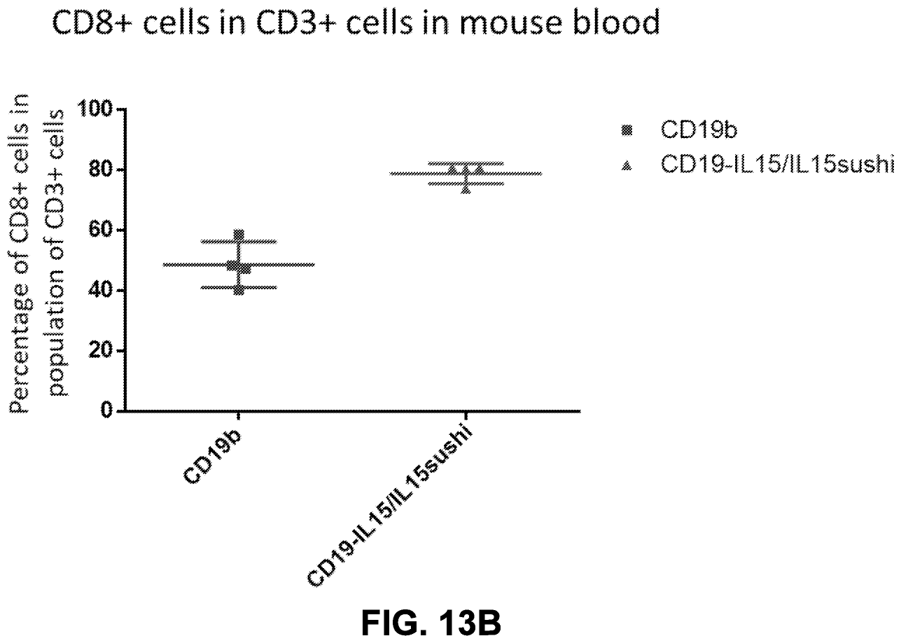

The overall persistence of T cells in mouse blood from the model in FIG. 12C was assayed at survival endpoints and screened by flow cytometry using CD3 antibody for bulk T cell populations (FIG. 13A). To further dissect the persistency results of the CD19b-IL-15/IL-15sushi armored CAR, the collection of mouse blood is necessary to reveal the presence of durability of the engrafted human cells. Overall, we found by flow cytometry analysis that there was a higher average count of T cells in the armored CAR cohorts when compared to the standard CART19 groups. Control group T cells remained at baseline as expected due to minimal stimulation from circulating in vivo tumor.

Mouse blood from FIG. 12C was furthered analyzed in FIG. 13B by CD8 expression in CD3 positive subsets to reveal the degree of persistent cytotoxic T cells remaining in circulation at survival endpoints. Of particular note is the much higher amount of cytotoxic CD8+ T cells present in the armored CAR (IL-15-/IL-15sushi) cohort mice blood, signifying that the expansion of tumor-killing T cells was greatly augmented not just by signal transduction from standard target engagement, but also by the inclusion of the IL-15 based cytokine secretory complex “armor.” Comparison to the standard CAR CD19 cohort shows the standard response expected from CAR therapy with the expansion of cells solely accomplished by target engagement and subsequent signal response.

### Examples of Immune Cells Expressing IL-15/IL-15sushi and CCL19 Exhibit Significant Anti-Tumor Activity, and Greater Persistence than Immune Cells Expressing IL-15/IL-15sushi, in the Xenogeneic Mouse Model

The invention disclosed immune cells, T cells expressing IL-15/IL-15sush and CCL19 with a CAR, CD19 CAR. This construct is called CD19b-XX which has a CD19 CAR and secreting IL-15/IL-15sushi and CCL-19. This system allows us to test how the combination of SCCL-19 and IL-15/IL-15sushi enhances immune cell functions in vivo.

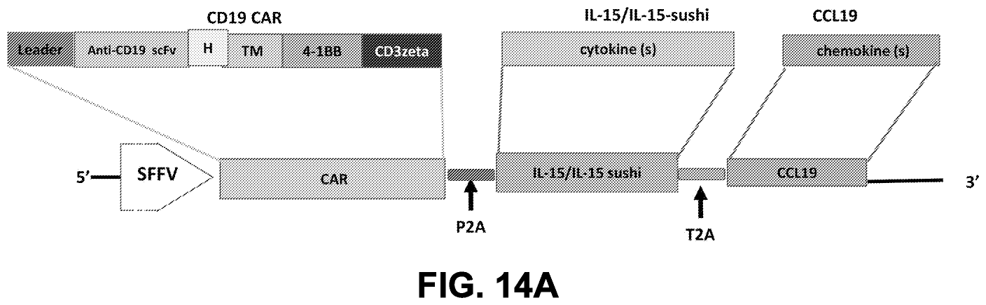

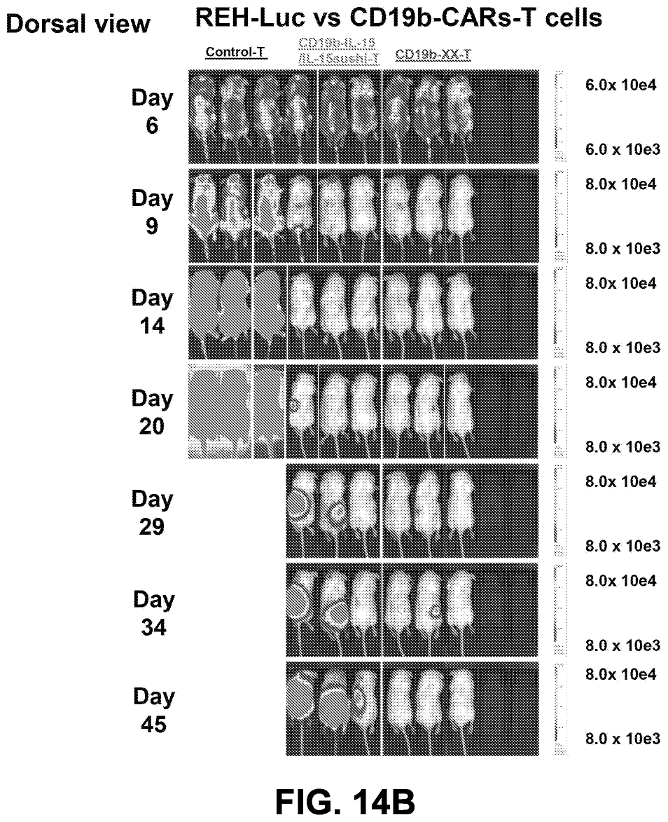

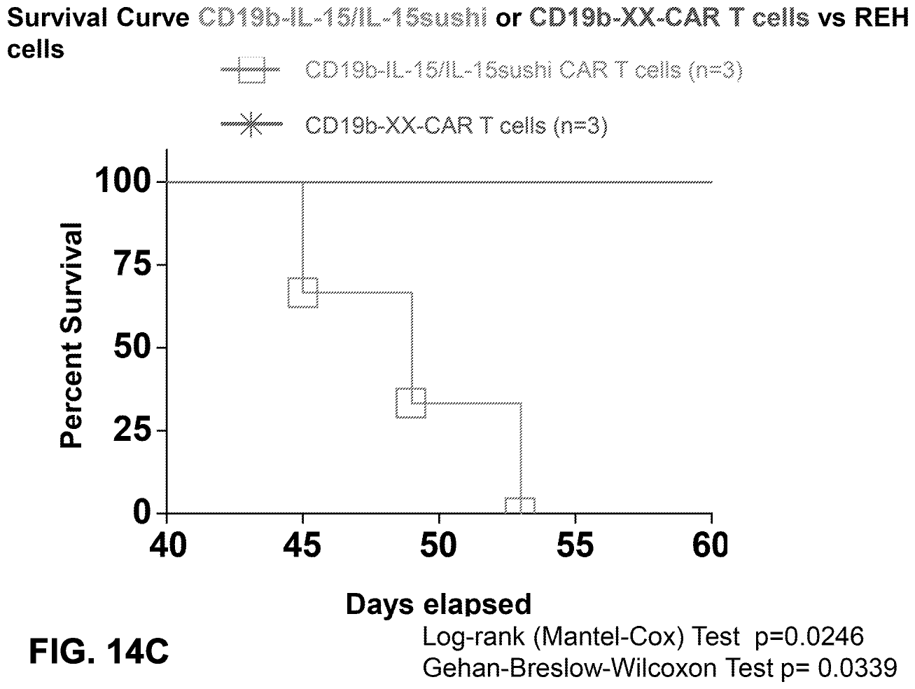

A schematic (FIG. 14A) showing a CD19-Q-XX CAR equipped with a cytokine complex, IL-15/IL-15sushi and a chemokine, CCL19. Activated human peripheral blood T cells were transduced with the lentiviral vector from CD19b-XX or CD19b-IL-15/IL-15 sushi CAR. The transduction efficiency between activated T cells transduced with either control vector, or CD19b-IL-15/IL-15/sushi or CD19b-XX CAR construct, as determined by labeling with goat anti-mouse F(Ab′)2 antibody. Activated T cells transduced with the CAR vectors resulted in 60% F(Ab′)2 positive cells for CD19b-IL-15/IL-15/sushi, and 58% F(Ab′)2 positive cells for CD19b-XX four days after the start of transduction.

Both CD19b-IL-15/IL-15sushi and CD19b-XX-CAR-T-cells completely lyse target REH cells in vitro 24 h co-culture assay—CD19b-IL-15/IL-15sushi and CD19b-XX-CAR-T-cells were assayed for their ability to specifically lyse REH tumor cells expressing CD19 antigen. Co-cultures with either control T cells. CD19b-IL-15/IL-15sushi or CD19b-XX CAR T cells against REH tumor cells at both 2:1 and 5:1 effector cell:target cell ratio, for 24 hours. Following this incubation, cells were stained using mouse anti-human CD3 and CD19 antibodies and analyzed by flow cytometry. After co-culture, nearly all of the tumor cells were lysed at both ratios. These results demonstrate that both CD19b-IL-15/IL-15sushi and CD19b-XX CAR T cells are equally effective at completely lysing their intended target cells.
Function of IL15/IL-15sushi in CD19b-XX CAR NK cells—To determine if IL-15/IL-15sushi is being secreted, the IL-15 dependent NK-92 cell line was transduced with lentiviral vector containing CD19b-XX CAR. Cells were sorted on BD FACS Aria to select NK cells positive for the F(Ab′)2 (CAR) phenotype. Sorted cells were expanded and labeled with goat anti-mouse F(Ab′)2 antibody and analyzed by flow cytometry to confirm the cells were nearly 100% positive for CAR phenotype.
IL-15/IL-15sushi secreted from CD19b-XX CAR NK cells can substitute for the function of IL-2 in vitro. Sorted CD19b-XX CAR NK cells, and wild-type NK-92 cells, were cultured in a 24-well plate at 0.5×10e6 cells per mL, in 1 mL total volume. Cells were added to duplicate wells; one well of each pair contained IL-2 at 300 IU/mL, the other well did not. After 48 hours (Day 2), cells were counted, and the volume increased to yield a concentration of approximately 0.5×10e6 cells/mL. This process was repeated on Days 4 and 6. CD19b-XX NK CAR T cells cultured for 6 days without IL-2 in the culture expanded at close to the same rate as wild-type NK-92 cells cultured with IL-2 added, whereas wild-type NK-92 cultured without IL-2 had all died by Day 6. This indicates that IL-15 secreted by the NK CAR cells can substitute for the expansion activity of IL-2.
CD19b-XX-CAR-T-cells cells exhibit significant anti-tumor activity, and greater persistence than CD19b-IL-15/IL-15sushi CAR T cells, in xenogeneic mouse model—In order to evaluate the specific in vivo anti-tumor activity of CD19b-IL-15/IL-15sushi (co-expressing IL-15/IL-15sushi) and CD19b-XX-CAR-T-cells (co-expressing IL-15/IL-15sush plus CCL9) against human tumor cell lines, we developed a xenogeneic mouse model using NSG mice sublethally irradiated and intravenously injected with 1×10⁶ of luciferase-expressing REH wild type acute myeloid leukemia tumor cells, which express CD19 on the cell surface, to induce measurable tumor formation. Seven days following tumor cell injection, all mice were intravenously injected with a course of a low dose, ˜0.3×10⁶ of either control T cells or CD19b-IL-15/IL-15sushi or CD19b-XX CAR T cells. On Day 6 (the day before T cell treatment), day 9 (48 hours after T cell treatment), and periodically thereafter, mice were subjected to IVIS imaging to measure tumor burden (FIG. 14B). Average light intensity measured for the REH mice injected with CD19b-IL-15/IL-15sushi or CD19b-XX CAR T cells was compared to that of mice injected with the control T cells to determine percent lysis of targeted cells. Results showed that only 3 days following treatment with T cells (Day 9), mice treated with either CAR T cells had far lower tumor burden than mice given control T cells (FIG. 14B). By Day 27, all three control mice had died. However, by Day 21, tumor cells began to expand in mice treated with CD19b-IL-15/IL-15sushi CAR T cells, relative to mice treated with CD19b-XX CAR T cells. By Day 45, mice treated with CD19b-IL-15/IL-15sushi CAR T cells had considerably more tumor cells than mice treated with CD19b-XX CAR T cells. While the CD19b-IL-15/IL-15sushi CAR T cells treated mice died by Day 53, mice treated with CD19b-XX CAR T cells survived at least until Day 60 (p=0.02) (FIG. 14C). These results show the increased efficacy and long-term effects CD19b-XX CAR T compared to CD19b-IL-15/IL-15sushi CAR T cells against a B-ALL tumor cell line in vivo.

It is unexpected that co-expression of one of chemokines including CCL19 and IL-15/IL-15sushi with a CAR is a very strong strategy for cancer treatment. This novel approach provides a long-term durable remission (FIGS. 14B and 14C).

The invention is also based on unexpected findings in mice that combination of CAR co-expressing IL-15/IL-15sushi and CCL19 provides a more effective anti-tumor response than CAR co-expressing IL-15/IL-sushi alone.

A similar strategy is expected that co-expression of one of chemokines including CCL19 and IL-15/IL-15sushi is a very strong strategy for an immune cell treating a cancer and an infectious disease.

In a clinical trial of T cells co-expressing CD19 CAR and IL-15/IL-15sushi (T or NK cell enhancer), in patients with B-cell acute lymphoblastic leukemia, it was surprisingly found that patients infused with these T cells only produced a picogram-level IL-15/IL-15sushi, and there was no manifestation of abnormal T cell proliferation. In addition, after 2.5 years of follow-up observation in the human clinical trial, CD19 IL-15/IL-15 sushi CAR showed excellent curative effects, with higher complete remission rate, and there was no evidence of T cell autonomous growth or leukemia occurred (see Table I).

TABLE 1
Patient Characteristic and Responses

| | | Prior | BM | Cytogenetic/ | CAR-T | | Bridge to | Time of | | |
|---|---|---|---|---|---|---|---|---|---|---|
| Patient | Age/sex | treatment | Blast % | molecular | Dose | responses | HSCT | follow up | | Current status |
| P1 | 11/F | 11 Chemo | 93% | NK | 0.8 × 10⁶/kg | CR/MRD− | NO | 30 | M | LFS |
| | | allo-HCT | | | | | | | | |
| P2 | 8/F | 11 chemo | 91% | T(1; 19) | 0.8 × 10⁶/kg | CR/MRD− | NO | 27 | M | LFS |
| | | | | E2A/PBX1 | | | | | | |
| P3 | 49/M | 15 chemo | 4% | t(9; 22) | 1.3 × 10⁶/kg | CR/MRD− | NO | 27 | M | LFS |
| | | | | BCR/ABL | | | | | | |
| P4 | 16/M | 5 chemo | 3% | NK | 1 × 10⁶/kg | CR/MRD− | YES | 23 | M | Relapsed |
| | | | | | | | | | | after BMT |
| P5 | 10/M | 11 chemo | 67.5% | NA | 1.3 × 10⁶/kg | CR/MRD− | YES | 174 | D | Died of GVHD |
| | | | | Tel/AML1 | | | | | | |
| P6 | 4/F | 4-chemo | 5% | NK | 1 × 10⁶/kg | CR/MRD− | NO | 23 | M | LFS |
| | | Haplo-HCT | | | | | | | | |
| P7 | 50/M | 13 chemo | 12% | NK | 0.8 × 10⁶/kg | CR/MRD− | NO | 23 | D | Lost follow up |
| P8 | 22/M | 18 chemo | 19.23% | NK | 1.1 × 10⁶/kg | CR/MRD− | YES | 8 | M | Relapsed |
| | | | | | | | | | | after BMT |
| P9 | 5/F | 11 chemo | 18% | NA | 3 × 10⁶/kg | CR/MRD− | NO | 3 | M | Relapsed at |
| | | | | | | | | | | 2 M |
| P10 | 17/M | Allo-HSCT | 19.23% | NK | 1 × 10⁶/kg | CR/MRD− | NO | 8 | M | LFS |
| P11 | 17/F | 12 chemo | 5.5% | NK | 1.25 × 10⁶/kg | CR/MRD− | NO | 2 | M | LFS |
| P12 | 34/F | Allo-HSCT | 1.15 | t(9; 22) | 1 × 10⁶/kg | CR/MRD− | NO | 1 | M | LFS |

BCR/ABL

B-ALL, B-cell acute lymphoblastic leukemia; NK, normal karyotype; NA, none available; LFS, Leukemia free survive; CR, complete remission; MRD−; minimal residual disease negative; None of Patients after treating with CD19 CAR co-expressing IL15/IL15sush developed abnormal T cell growth detected.

### CD19b-RTX-TM, CD19b-IL15/IL15sushi-RTX-TM, and CD19b-RTX-7-TM CAR T Cells Express CAR and Rituximab in Transduced Human T Cells

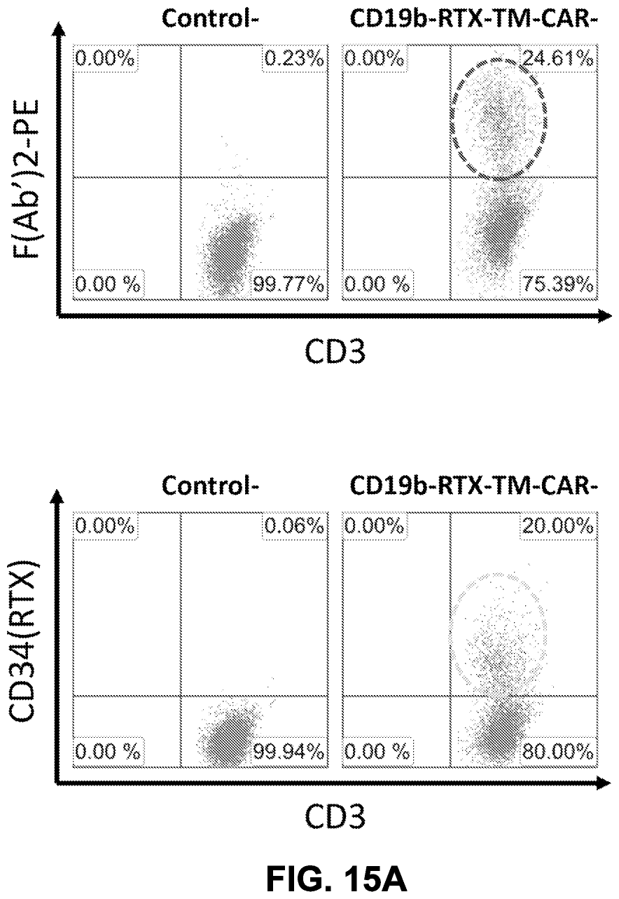

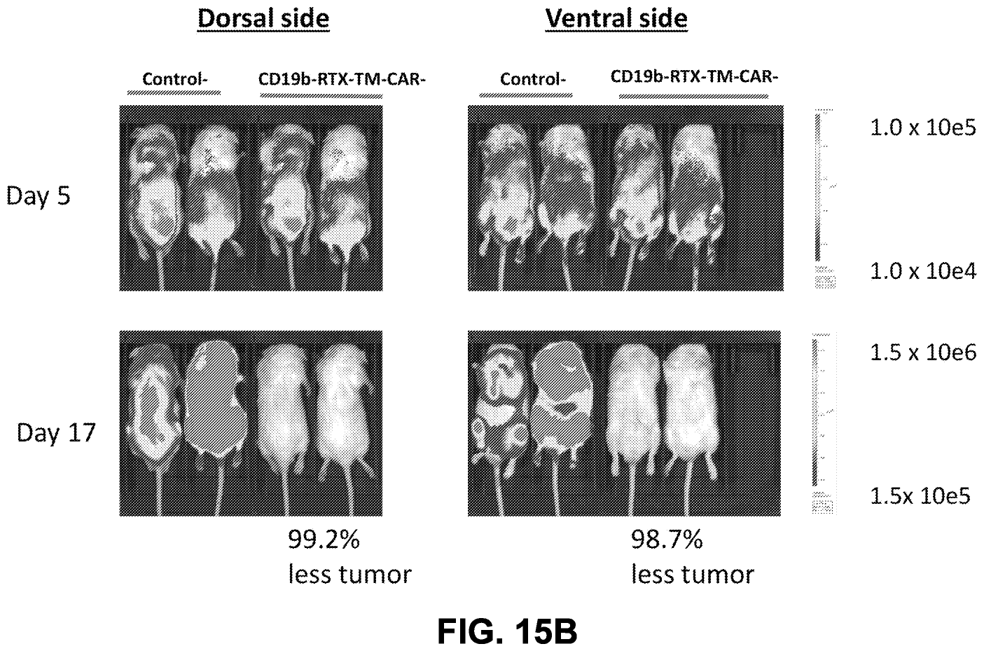

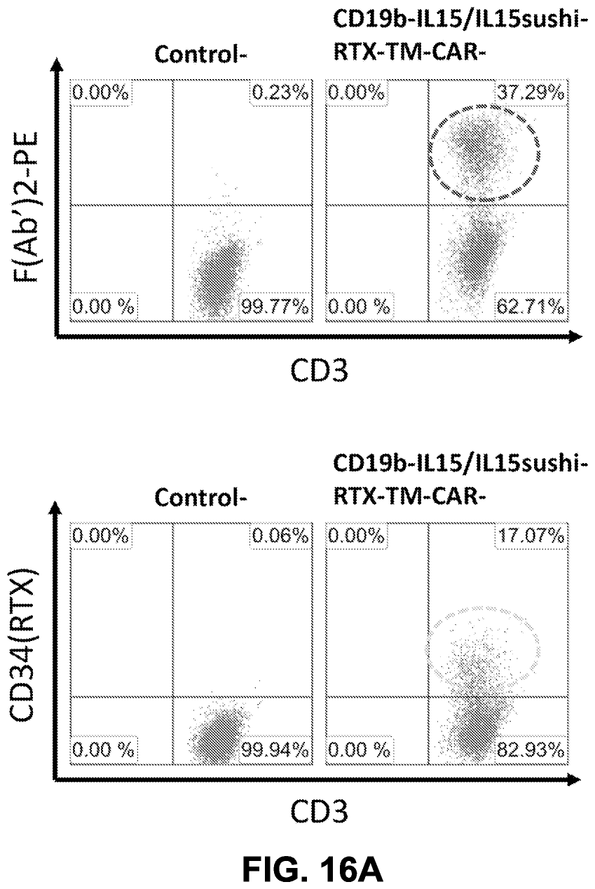

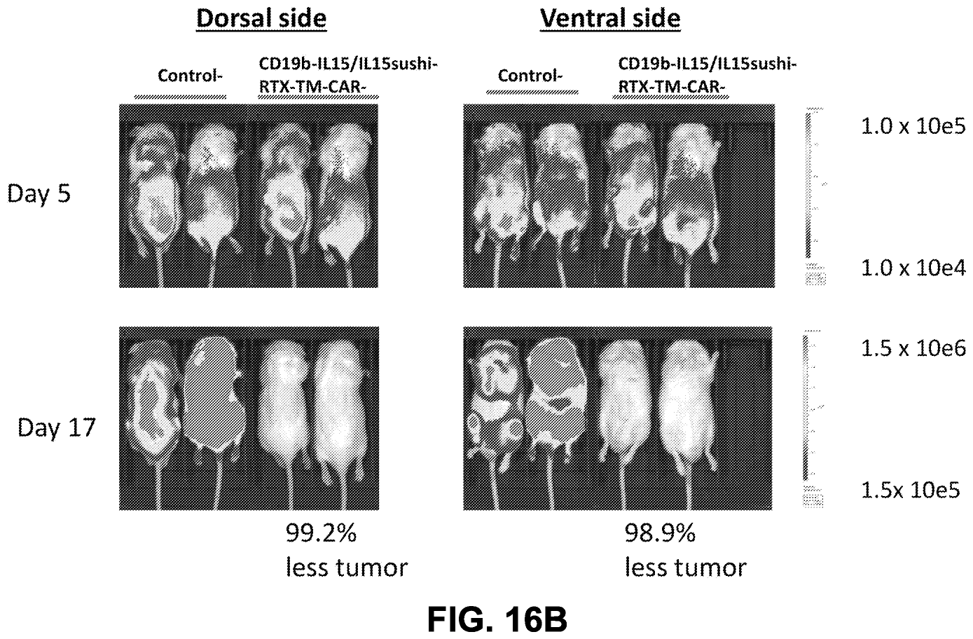

Activated human T cells were transduced with of CD19b-RTX-TM, CD19b-IL15/IL15sushi-RTX-TM, and CD19b-RTX-7-TM CAR lentiviral vector. To determine the percent CAR efficiency (surface expression) of the transductions, and to detect the presence of the safety switch (rituximab), transduced cells were labeled with goat anti-mouse F(Ab′)2 and CD34 (RTX) antibodies and analyzed by flow cytometry. Results show that approximately 24.6% of cells transduced with CD19b-RTX-TM lentiviral vector were CAR cells (FIG. 15A), 37.3% of the T cells transduced with CD19b-IL15/IL15sushi-RTX-TM lentiviral vector were CAR cells (FIG. 16A), and 32.3% of the T cells transduced with CD19b-RTX-7-TM lentiviral vector were CAR cells (FIG. 17A).

Approximately 20% of cells transduced with CD19b-RTX-TM lentiviral vector were positive for CD34, and therefore expressed the rituximab safety switch cells (FIG. 15A), 17% of the T cells transduced with CD19b-IL15/IL15sushi-RTX-TM lentiviral vector were CD34+ (FIG. 16A), and 25% of the T cells transduced with CD19b-RTX-7-TM lentiviral vector were CD34+ cells (FIG. 17A). Therefore, all three sets of transduced T cells expressed both CAR and rituximab phenotypes and could be used in the in vivo assays.

### CD19b-RTX-TM, CD19b-IL15/IL15sushi-RTX-TM, and on CD19b-RTX-7-TM CAR T Cells Exhibit Significant Anti-Tumor Activity in Vivo in a B-ALL Xenograft Mouse Model

In order to evaluate the in vivo anti-tumor activity of CD19b-RTX-TM, CD19b-IL15/IL15sushi-RTX-TM, and CD19b-RTX-7-TM CAR T cells, we developed a xenograft mouse model using NSG mice sublethally (2.0 Gy) gamma irradiated and intravenously injected with 1.0×10⁶ firefly luciferase-expressing REH cells (a B cell acute lymphoblastic leukemia cell line) to induce measurable tumor formation. On day 6, 5 days following REH-luciferase cell injection, mice were intravenously injected with 10×10⁶ of either CAR T cells (CD19b-RTX-TM, CD19b-IL15/IL15sushi-RTX-TM, or CD19b-RTX-7-TM or control T cells. On days 5 (before CAR T treatment), 8, 11, 14 and 17, mice were injected subcutaneously with RediJect D-Luciferin and subjected to IVIS imaging to measure tumor burden (FIGS. 15B, 16B and 17B). Average light intensity measured for CAR T cell injected mice was compared to that of the control T cell injected mice.

Total flux levels continually increased in control mice with drastic tumor burden growth on both dorsal and ventral sides (FIG. 15B). Compared to control mice, CD19b-RTX-TM-CAR treated mice showed 76.9% (day 11), 94.2% (day 14) and nearly complete (99.2%; day 17) tumor suppression in dorsal side. 73.5% (day 11), 92.6% (day 14) and nearly complete (98.7%; day 17) of tumor suppression was seen in ventral side (FIG. 15B).

CD19b-IL15/IL15sushi-RTX-TM-CAR T cells treated mice showed 77.1% (day 11), 93.8% (day 14) and nearly complete (99.2%; day 17) tumor suppression in dorsal side (FIG. 16B). 73.1% (day 11), 92.8% (day 14) and 98.9% (day 17) of tumor suppression were seen in ventral side (FIG. 16B).

CD19b-RTX-7-TM-CAR T cells treated mice showed 77.7% (day 11), 93.9% (day 14) and nearly complete (99.2%; day 17) tumor suppression in dorsal side (FIG. 17B). 71.9% (day 11), 92.1% (day 14) and 98.8% (day 17) of tumor suppression were seen in ventral side (FIG. 17B).

In summary, these in vivo data indicate that all three CARs—CD19b-RTX-TM, CD19b-IL15/IL15sushi-RTX-TM, and CD19b-RTX-7-TM CAR T cells significantly reduce tumor burden in REH-injected NSG mice when compared to control T control cells.

### INCORPORATION OF SEQUENCE LISTING

Incorporated herein by reference in its entirety is the Sequence Listing for the application. The Sequence Listing is disclosed on a computer-readable ASCII text file titled, “sequence_listing.txt”.

1-22. (canceled)

23. An engineered immunoregulatory cell comprising at least one enhancer, wherein said enhancer comprises IL-15/IL-15sushi, IL-15/IL-15sushi anchor, PD-1, PD-L1, CSF1R, CTAL-4, TIM-3, TGFR beta, IL-2, IL-7, IL-12, IL-15, CCL-19, CCL-21, IL-15RA, IL-21, or functional fragments thereof, or combinations thereof, and wherein the engineered immunoregulatory cell binds to a target cell.

24. The engineered cell according to claim 23, wherein the enhancer comprises IL-15/IL-15sushi.

25. The engineered cell according to claim 23, wherein the enhancer comprises IL-15/IL-15sushi and IL-15/IL-15sushi anchor.

26. The engineered cell according to claim 23, wherein the enhancer comprises IL-15 and IL-15/IL-15sushi anchor.

27. The engineered cell according to claim 23, wherein the enhancer comprises IL-7 and IL-15/IL-15sushi anchor.

28. The engineered cell according to claim 23, wherein the enhancer comprises IL-15/IL-15sushi, IL-15/IL-15sushi anchor and CCL-19.

29. The engineered cell according to claim 23, wherein the enhancer comprises IL-7, IL-15/IL-15sushi anchor and CCL-19.

30. The engineered cell according to claim 23, wherein the enhancer comprises IL-7, IL-15 and CCL-19.

31. The engineered cell according to claim 23, wherein the enhancer comprises IL-15/IL-15sushi and CCL-19.

32. The engineered cell according to claim 23, wherein the enhancer comprises IL-15/IL-15sushi anchor and CCL-19.

33. The engineered cell according to claim 23, wherein the enhancer comprises IL-7 and IL-15

34. The engineered cell according to claim 23, wherein the enhancer comprises IL-15/IL-15sushi anchor.

35. The engineered cell according to claim 23, wherein said engineered cell comprises a T-Cell, NK cell, MR1-R T cell, NK T cell, gamma delta (γδ) T cell or macrophage.

36. The engineered cell according to claim 23, wherein said target cell is a cancer cell.

37. The engineered cell according to claim 23, wherein said target cell is a lymphoma cell or leukemia cell.

38. The engineered cell according to claim 23, wherein said target cell is a cell infected with a virus, bacterium, fungi or parasite.

39. The engineered cell according to claim 38, wherein said virus is herpes simplex virus (HSV), Epstein-Barr virus (EBV), varicella Zoster virus (VZV), cytomegalovirus (CMV), human papilloma virus (HPV), human immunodeficiency virus (HIV), or a coronavirus.

40. The engineered cell according to claim 39, wherein said coronavirus is middle eastern respiratory syndrome (MERS), severe acute respiratory syndrome (SARS) or COVID-19.

41. The engineered cell according to claim 40, wherein said target cell is an infected cell with COVID-19.


SEQUENCE LISTING (20 sequences)

SEQ ID NO: 1 - protein, 715 aa, Artificial Sequence
  synthetic sequence
MALPVTALLLPLALLLHAARPQVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 2 - DNA, 2148 bp, Artificial Sequence
  synthetic sequence
atggccttaccagtgaccgccttgctcctgccgctggccttgctgctccacgccgccaggccgcaagtgcaactgcagcagagcggcccagaggtggtcaaacctggggcaagcgtgaagatgagctgcaaggctagcggctataccttcaccagctatgtgatccactgggtgaggcagaaaccaggacagggcctggactggatcggctacatcaacccctacaatgacggcaccgattatgacgaaaaattcaaggggaaggccaccctgaccagcgacaccagcacaagcaccgcctacatggagctgtccagcctgaggtccgaggacaccgccgtgtattactgtgccagggagaaggacaattacgccaccggcgcttggttcgcctactggggccagggcacactggtgacagtgagcagcgcctgcccctacagcaaccccagcctgtgcagcggcggcggcggcagcgagctgcccacccagggcaccttcagcaacgtgagcaccaacgtgagccccgccaagcccaccaccaccgcctgtccttattccaatccttccctgtgtagcggcggcggcggcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggcagcggcgaaggccgcggcagcctgctgacctgcggcgatgtggaagaaaacccgggccccatgtacagaatgcagctgctgagctgcatcgccctgagcctggccctggtgaccaacagcggcatccacgtgttcatcctgggctgcttcagcgccggcctgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagctccggcggcggctccggcggcggcggctccggcggcggcggctccggcggcggcggctccggcggcggctccctgcaggcccccagaagagccagaggctgcagaaccctgggcctgcccgccctgctgctgctgctgctgctgagaccccccgccaccagaggcatcacctgccccccccccatgagcgtggagcacgccgacatctgggtgaagagctacagcctgtacagcagagagagatacatctgcaacagcggcttcaagagaaaggccggcaccagcagcctgaccgagtgcgtgctgaacaaggccaccaacgtggcccactggaccacccccagcctgaagtgcatcagataa

SEQ ID NO: 3 - protein, 771 aa, Artificial Sequence
  synthetic sequence
MALPVTALLLPLALLLHAARPEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCGSGATNFSLLKQAGDVEENPGPMFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGSGEGRGSLLTCGDVEENPGPMYRMQLLSCIALSLALVTNSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRTTTPAPRPPTPAPTIASQPLSLRPEACRPAAGGAVHTRGLDFACDVAISTSTVLLCGLSAVSLLACYL

SEQ ID NO: 4 - DNA, 2316 bp, Artificial Sequence
  synthetic sequence
atggccttaccagtgaccgccttgctcctgccgctggccttgctgctccacgccgccaggccggaggtgcagctgctgcagagcggccccgagctggagaagcccggcgccagcgtgatgatcagctgcaaggccagcggcagcagcttcaccggctacaacatgaactgggtgaggcagaacatcggcaagagcctggagtggatcggcgccatcgacccctactacggcggcaccagctacaaccagaagttcaagggcagggccaccctgaccgtggacaagagcagcagcaccgcctacatgcacctgaagagcctgaccagcgaggacagcgccgtgtactactgcgtgagcggcatggagtactggggccagggcaccagcgtgaccgtgagcagcgcctgcccctacagcaaccccagcctgtgcagcggcggcggcggcagcgagctgcccacccagggcaccttcagcaacgtgagcaccaacgtgagccccgccaagcccaccaccaccgcctgtccttattccaatccttccctgtgtagcggcggcggcggcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcggaagcggagccaccaacttcagcctgctgaagcaggccggcgacgtggaggagaaccccggccccatgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacacggcagcggcgaaggccgcggcagcctgctgacctgcggcgatgtggaagaaaacccgggccccatgtacagaatgcagctgctgagctgcatcgccctgagcctggccctggtgaccaacagcaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagctccggcggcggctccggcggcggcggctccggcggcggcggctccggcggcggcggctccggcggcggctccctgcagatcacctgccccccccccatgagcgtggagcacgccgacatctgggtgaagagctacagcctgtacagcagagagagatacatctgcaacagcggcttcaagagaaaggccggcaccagcagcctgaccgagtgcgtgctgaacaaggccaccaacgtggcccactggaccacccccagcctgaagtgcatcagaaccaccacccccgcccccaggccccccacccccgcccccaccatcgccagccagcccctgagcctgaggcccgaggcctgcaggcccgccgccggcggcgccgtgcacaccaggggcctggacttcgcctgcgacgtggctatctccacgtccactgtcctgctgtgtgggctgagcgctgtgtctctcctggcatgctacctgtaa

SEQ ID NO: 5 - protein, 827 aa, Artificial Sequence
  synthetic sequence
ALPVTALLLPLALLLHAARPEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCGSGATNFSLLKQAGDVEENPGPMRISKPHLRSISIQCYLCLLLNSHFLTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRGSGEGRGSLLTCGDVEENPGPMYRMQLLSCIALSLALVTNSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRTTTPAPRPPTPAPTIASQPLSLRPEACRPAAGGAVHTRGLDFACDVAISTSTVLLCGLSAVSLLACYL

SEQ ID NO: 6 - DNA, 2487 bp, Artificial Sequence
  synthetic sequence
atggccttaccagtgaccgccttgctcctgccgctggccttgctgctccacgccgccaggccggaggtgcagctgctgcagagcggccccgagctggagaagcccggcgccagcgtgatgatcagctgcaaggccagcggcagcagcttcaccggctacaacatgaactgggtgaggcagaacatcggcaagagcctggagtggatcggcgccatcgacccctactacggcggcaccagctacaaccagaagttcaagggcagggccaccctgaccgtggacaagagcagcagcaccgcctacatgcacctgaagagcctgaccagcgaggacagcgccgtgtactactgcgtgagcggcatggagtactggggccagggcaccagcgtgaccgtgagcagcgcctgcccctacagcaaccccagcctgtgcagcggcggcggcggcagcgagctgcccacccagggcaccttcagcaacgtgagcaccaacgtgagccccgccaagcccaccaccaccgcctgtccttattccaatccttccctgtgtagcggcggcggcggcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcggaagcggagccaccaacttcagcctgctgaagcaggccggcgacgtggaggagaaccccggccccatgagaatttcgaaaccacatttgagaagtatttccatccagtgctacttgtgtttacttctaaacagtcattttctaactgaagctaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctagcggcggcggcagcggtggtggcggtagcggcggtggtggcagcggcggtggcggtagcggtggcggtagcctgcagattacctgtccgccgccgatgtccgttgaacatgcggatatttgggttaaatcctatagcctgtatagccgcgaacgctacatctgtaatagcggttttaaacgcaaagcgggcacctccagcctgaccgaatgtgtgctgaataaagcgaccaatgttgcgcattggaccaccccgagcctgaaatgtattcgcggcagcggcgaaggccgcggcagcctgctgacctgcggcgatgtggaagaaaacccgggccccatgtacagaatgcagctgctgagctgcatcgccctgagcctggccctggtgaccaacagcaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagctccggcggcggctccggcggcggcggctccggcggcggcggctccggcggcggcggctccggcggcggctccctgcagatcacctgccccccccccatgagcgtggagcacgccgacatctgggtgaagagctacagcctgtacagcagagagagatacatctgcaacagcggcttcaagagaaaggccggcaccagcagcctgaccgagtgcgtgctgaacaaggccaccaacgtggcccactggaccacccccagcctgaagtgcatcagaaccaccacccccgcccccaggccccccacccccgcccccaccatcgccagccagcccctgagcctgaggcccgaggcctgcaggcccgccgccggcggcgccgtgcacaccaggggcctggacttcgcctgcgacgtggctatctccacgtccactgtcctgctgtgtgggctgagcgctgtgtctctcctggcatgctacctgtaa

SEQ ID NO: 7 - protein, 1066 aa, Artificial Sequence
  synthetic sequence
MALPVTALLLPLALLLHAARPEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGEGRGSLLTCGDVEENPGPMAPRRARGCRTLGLPALLLLLLLRPPATRGGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDVAISTSTVLLCGLSAVSLLACYL

SEQ ID NO: 8 - DNA, 3201 bp, Artificial Sequence
  synthetic sequence
atggccttaccagtgaccgccttgctcctgccgctggccttgctgctccacgccgccaggccggaggtccagctgcagcagtctggacctgagctgataaagcctggggcttcagtgaagatgtcctgcaaggcttctggatacacattcactagctatgttatgcactgggtgaagcagaagcctgggcagggccttgagtggattggatatattaatccttacaatgatggtactaagtacaatgagaagttcaaaggcaaggccacactgacttcagacaaatcctccagcacagcctacatggagctcagcagcctgacctctgaggactctgcggtctattactgtgcaagagggacttattactacggtagtagggtatttgactactggggccaaggcaccactctcacagtctcctcaggtggagggggctcaggcggaggtggctctgggggtggaggctcggacattgtgatgactcaggctgcaccctctatacctgtcactcctggagagtcagtatccatctcctgcaggtctagtaagagtctcctgaatagtaatggcaacacttacttgtattggttcctgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatccgttcacgttcggtgctgggaccaagctggagctgaaacggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagccaccaacttcagcctgctgaagcaggccggcgacgtggaggagaaccccggccccatgtacagaatgcagctgctgagctgcatcgccctgagcctggccctggtgaccaacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctggcagcggcgaaggccgcggcagcctgctgacctgcggcgatgtggaagaaaacccgggccccatggcccccagaagagccagaggctgcagaaccctgggcctgcccgccctgctgctgctgctgctgctgagaccccccgccaccagaggcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttccggcggcggctccggcggcggcggctccggcggcggcggctccggcggcggcggctccggcggcggctccctgcagatcacctgccccccccccatgagcgtggagcacgccgacatctgggtgaagagctacagcctgtacagcagagagagatacatctgcaacagcggcttcaagagaaaggccggcaccagcagcctgaccgagtgcgtgctgaacaaggccaccaacgtggcccactggaccacccccagcctgaagtgcatcagagcctgcccctacagcaaccccagcctgtgcagcggcggcggcggcagcgagctgcccacccagggcaccttcagcaacgtgagcaccaacgtgagccccgccaagcccaccaccaccgcctgtccttattccaatccttccctgtgtagcggcggcggcggcagcaccaccacccccgcccccaggccccccacccccgcccccaccatcgccagccagcccctgagcctgaggcccgaggcctgcaggcccgccgccggcggcgccgtgcacaccaggggcctggacttcgcctgcgacgtggctatctccacgtccactgtcctgctgtgtgggctgagcgctgtgtctctcctggcatgctacctgtaa

SEQ ID NO: 9 - protein, 892 aa, Artificial Sequence
  synthetic sequence
MALPVTALLLPLALLLHAARPEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMAPRRARGCRTLGLPALLLLLLLRPPATRGGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDVAISTSTVLLCGLSAVSLLACYL

SEQ ID NO: 10 - DNA, 2679 bp, Artificial Sequence
  synthetic sequence
atggccttaccagtgaccgccttgctcctgccgctggccttgctgctccacgccgccaggccggaggtccagctgcagcagtctggacctgagctgataaagcctggggcttcagtgaagatgtcctgcaaggcttctggatacacattcactagctatgttatgcactgggtgaagcagaagcctgggcagggccttgagtggattggatatattaatccttacaatgatggtactaagtacaatgagaagttcaaaggcaaggccacactgacttcagacaaatcctccagcacagcctacatggagctcagcagcctgacctctgaggactctgcggtctattactgtgcaagagggacttattactacggtagtagggtatttgactactggggccaaggcaccactctcacagtctcctcaggtggagggggctcaggcggaggtggctctgggggtggaggctcggacattgtgatgactcaggctgcaccctctatacctgtcactcctggagagtcagtatccatctcctgcaggtctagtaagagtctcctgaatagtaatggcaacacttacttgtattggttcctgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatccgttcacgttcggtgctgggaccaagctggagctgaaacggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagccaccaacttcagcctgctgaagcaggccggcgacgtggaggagaaccccggccccatggcccccagaagagccagaggctgcagaaccctgggcctgcccgccctgctgctgctgctgctgctgagaccccccgccaccagaggcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttccggcggcggctccggcggcggcggctccggcggcggcggctccggcggcggcggctccggcggcggctccctgcagatcacctgccccccccccatgagcgtggagcacgccgacatctgggtgaagagctacagcctgtacagcagagagagatacatctgcaacagcggcttcaagagaaaggccggcaccagcagcctgaccgagtgcgtgctgaacaaggccaccaacgtggcccactggaccacccccagcctgaagtgcatcagagcctgcccctacagcaaccccagcctgtgcagcggcggcggcggcagcgagctgcccacccagggcaccttcagcaacgtgagcaccaacgtgagccccgccaagcccaccaccaccgcctgtccttattccaatccttccctgtgtagcggcggcggcggcagcaccaccacccccgcccccaggccccccacccccgcccccaccatcgccagccagcccctgagcctgaggcccgaggcctgcaggcccgccgccggcggcgccgtgcacaccaggggcctggacttcgcctgcgacgtggctatctccacgtccactgtcctgctgtgtgggctgagcgctgtgtctctcctggcatgctacctgtaa

SEQ ID NO: 11 - protein, 1079 aa, Artificial Sequence
  synthetic sequence
MALPVTALLLPLALLLHAARPEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGSGEGRGSLLTCGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRTTTPAPRPPTPAPTIASQPLSLRPEACRPAAGGAVHTRGLDFACDVAISTSTVLLCGLSAVSLLACY

SEQ ID NO: 12 - DNA, 3240 bp, Artificial Sequence
  synthetic sequence
atggccttaccagtgaccgccttgctcctgccgctggccttgctgctccacgccgccaggccggaggtccagctgcagcagtctggacctgagctgataaagcctggggcttcagtgaagatgtcctgcaaggcttctggatacacattcactagctatgttatgcactgggtgaagcagaagcctgggcagggccttgagtggattggatatattaatccttacaatgatggtactaagtacaatgagaagttcaaaggcaaggccacactgacttcagacaaatcctccagcacagcctacatggagctcagcagcctgacctctgaggactctgcggtctattactgtgcaagagggacttattactacggtagtagggtatttgactactggggccaaggcaccactctcacagtctcctcaggtggagggggctcaggcggaggtggctctgggggtggaggctcggacattgtgatgactcaggctgcaccctctatacctgtcactcctggagagtcagtatccatctcctgcaggtctagtaagagtctcctgaatagtaatggcaacacttacttgtattggttcctgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatccgttcacgttcggtgctgggaccaagctggagctgaaacgggcctgcccctacagcaaccccagcctgtgcagcggcggcggcggcagcgagctgcccacccagggcaccttcagcaacgtgagcaccaacgtgagccccgccaagcccaccaccaccgcctgtccttattccaatccttccctgtgtagcggcggcggcggcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagctactaacttcagcctgctgaagcaggctggagacgtggaggagaaccctggacctatgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacacggcagcggcgaaggccgcggcagcctgctgacctgcggcgatgtggaagaaaacccgggccccatgtacagaatgcagctgctgagctgcatcgccctgagcctggccctggtgaccaacagcggcatccacgtgttcatcctgggctgcttcagcgccggcctgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagctccggcggcggctccggcggcggcggctccggcggcggcggctccggcggcggcggctccggcggcggctccctgcagatcacctgccccccccccatgagcgtggagcacgccgacatctgggtgaagagctacagcctgtacagcagagagagatacatctgcaacagcggcttcaagagaaaggccggcaccagcagcctgaccgagtgcgtgctgaacaaggccaccaacgtggcccactggaccacccccagcctgaagtgcatcagaaccaccacccccgcccccaggccccccacccccgcccccaccatcgccagccagcccctgagcctgaggcccgaggcctgcaggcccgccgccggcggcgccgtgcacaccaggggcctggacttcgcctgcgacgtggctatctccacgtccactgtcctgctgtgtgggctgagcgctgtgtctctcctggcatgctactaa

SEQ ID NO: 13 - protein, 747 aa, Artificial Sequence
  synthetic sequence
MALPVTALLLPLALLLHAARPEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 14 - DNA, 2244 bp, Artificial Sequence
  synthetic sequence
atggccttaccagtgaccgccttgctcctgccgctggccttgctgctccacgccgccaggccggaggtccagctgcagcagtctggacctgagctgataaagcctggggcttcagtgaagatgtcctgcaaggcttctggatacacattcactagctatgttatgcactgggtgaagcagaagcctgggcagggccttgagtggattggatatattaatccttacaatgatggtactaagtacaatgagaagttcaaaggcaaggccacactgacttcagacaaatcctccagcacagcctacatggagctcagcagcctgacctctgaggactctgcggtctattactgtgcaagagggacttattactacggtagtagggtatttgactactggggccaaggcaccactctcacagtctcctcaggtggagggggctcaggcggaggtggctctgggggtggaggctcggacattgtgatgactcaggctgcaccctctatacctgtcactcctggagagtcagtatccatctcctgcaggtctagtaagagtctcctgaatagtaatggcaacacttacttgtattggttcctgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatccgttcacgttcggtgctgggaccaagctggagctgaaacgggcctgcccctacagcaaccccagcctgtgcagcggcggcggcggcagcgagctgcccacccagggcaccttcagcaacgtgagcaccaacgtgagccccgccaagcccaccaccaccgcctgtccttattccaatccttccctgtgtagcggcggcggcggcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagctactaacttcagcctgctgaagcaggctggagacgtggaggagaaccctggacctatgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactaa

SEQ ID NO: 15 - protein, 881 aa, Artificial Sequence
  synthetic sequence
MALPVTALLLPLALLLHAARPEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRTTTPAPRPPTPAPTIASQPLSLRPEACRPAAGGAVHTRGLDFACDVAISTSTVLLCGLSAVSLLACY

SEQ ID NO: 16 - DNA, 2646 bp, Artificial Sequence
  synthetic sequence
atggccttaccagtgaccgccttgctcctgccgctggccttgctgctccacgccgccaggccggaggtccagctgcagcagtctggacctgagctgataaagcctggggcttcagtgaagatgtcctgcaaggcttctggatacacattcactagctatgttatgcactgggtgaagcagaagcctgggcagggccttgagtggattggatatattaatccttacaatgatggtactaagtacaatgagaagttcaaaggcaaggccacactgacttcagacaaatcctccagcacagcctacatggagctcagcagcctgacctctgaggactctgcggtctattactgtgcaagagggacttattactacggtagtagggtatttgactactggggccaaggcaccactctcacagtctcctcaggtggagggggctcaggcggaggtggctctgggggtggaggctcggacattgtgatgactcaggctgcaccctctatacctgtcactcctggagagtcagtatccatctcctgcaggtctagtaagagtctcctgaatagtaatggcaacacttacttgtattggttcctgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatccgttcacgttcggtgctgggaccaagctggagctgaaacgggcctgcccctacagcaaccccagcctgtgcagcggcggcggcggcagcgagctgcccacccagggcaccttcagcaacgtgagcaccaacgtgagccccgccaagcccaccaccaccgcctgtccttattccaatccttccctgtgtagcggcggcggcggcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagctactaacttcagcctgctgaagcaggctggagacgtggaggagaaccctggacctatgtacagaatgcagctgctgagctgcatcgccctgagcctggccctggtgaccaacagcggcatccacgtgttcatcctgggctgcttcagcgccggcctgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagctccggcggcggctccggcggcggcggctccggcggcggcggctccggcggcggcggctccggcggcggctccctgcagatcacctgccccccccccatgagcgtggagcacgccgacatctgggtgaagagctacagcctgtacagcagagagagatacatctgcaacagcggcttcaagagaaaggccggcaccagcagcctgaccgagtgcgtgctgaacaaggccaccaacgtggcccactggaccacccccagcctgaagtgcatcagaaccaccacccccgcccccaggccccccacccccgcccccaccatcgccagccagcccctgagcctgaggcccgaggcctgcaggcccgccgccggcggcgccgtgcacaccaggggcctggacttcgcctgcgacgtggctatctccacgtccactgtcctgctgtgtgggctgagcgctgtgtctctcctggcatgctactaa

SEQ ID NO: 17 - protein, 734 aa, Artificial Sequence
  synthetic sequence
MALPVTALLLPLALLLHAARPEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPMYRMQLLSCIALSLALVTNSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSTTTPAPRPPTPAPTIASQPLSLRPEACRPAAGGAVHTRGLDFACDVAISTSTVLLCGLSAVSLLACY

SEQ ID NO: 18 - DNA, 2221 bp, Artificial Sequence
  synthetic sequence
gcgatcgcatggccttaccagtgaccgccttgctcctgccgctggccttgctgctccacgccgccaggccggaggtccagctgcagcagtctggacctgagctgataaagcctggggcttcagtgaagatgtcctgcaaggcttctggatacacattcactagctatgttatgcactgggtgaagcagaagcctgggcagggccttgagtggattggatatattaatccttacaatgatggtactaagtacaatgagaagttcaaaggcaaggccacactgacttcagacaaatcctccagcacagcctacatggagctcagcagcctgacctctgaggactctgcggtctattactgtgcaagagggacttattactacggtagtagggtatttgactactggggccaaggcaccactctcacagtctcctcaggtggagggggctcaggcggaggtggctctgggggtggaggctcggacattgtgatgactcaggctgcaccctctatacctgtcactcctggagagtcagtatccatctcctgcaggtctagtaagagtctcctgaatagtaatggcaacacttacttgtattggttcctgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatccgttcacgttcggtgctgggaccaagctggagctgaaacggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggcagcggcgaaggccgcggcagcctgctgacctgcggcgatgtggaagaaaacccgggccccatgtacagaatgcagctgctgagctgcatcgccctgagcctggccctggtgaccaacagccagggccaggacaggcacatgatcaggatgaggcagctgatcgacatcgtggaccagctgaagaactacgtgaacgacctggtgcccgagttcctgcccgcccccgaggacgtggagaccaactgcgagtggagcgccttcagctgcttccagaaggcccagctgaagagcgccaacaccggcaacaacgagaggatcatcaacgtgagcatcaagaagctgaagaggaagccccccagcaccaacgccggcaggaggcagaagcacaggctgacctgccccagctgcgacagctacgagaagaagccccccaaggagttcctggagaggttcaagagcctgctgcagaagatgatccaccagcacctgagcagcaggacccacggcagcgaggacagcaccaccacccccgcccccaggccccccacccccgcccccaccatcgccagccagcccctgagcctgaggcccgaggcctgcaggcccgccgccggcggcgccgtgcacaccaggggcctggacttcgcctgcgacgtggctatctccacgtccactgtcctgctgtgtgggctgagcgctgtgtctctcctggcatgctactaagtttaaac

SEQ ID NO: 19 - protein, 1099 aa, Artificial Sequence
  synthetic sequence
DRTMALPVTALLLPLALLLHAARPDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIKGGGGSGGGGSGGGGSQIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSATTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSGEGRGSLLTCGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRTTTPAPRPPTPAPTIASQPLSLRPEACRPAAGGAVHTRGLDFACDVAISTSTVLLCGLSAVSLLACY

SEQ ID NO: 20 - DNA, 3299 bp, Artificial Sequence
  synthetic sequence
atggccttaccagtgaccgccttgctcctgccgctggccttgctgctccacgccgccaggccggacgtggtgatgacccagagccacaggttcatgagcaccagcgtgggcgacagggtgagcatcacctgcagggccagccaggacgtgaacaccgccgtgagctggtaccagcagaagcccggccagagccccaagctgctgatcttcagcgccagctacaggtacaccggcgtgcccgacaggttcaccggcagcggcagcggcgccgacttcaccctgaccatcagcagcgtgcaggccgaggacctggccgtgtactactgccagcagcactacagcaccccctggaccttcggcggcggcaccaagctggacatcaagggaggggggggatccgggggaggaggctccggcggaggcggaagccagatccagctggtgcagagcggccccgacctgaagaagcccggcgagaccgtgaagctgagctgcaaggccagcggctacaccttcaccaacttcggcatgaactgggtgaagcaggcccccggcaagggcttcaagtggatggcctggatcaacacctacaccggcgagagctacttcgccgacgacttcaagggcaggttcgccttcagcgtggagaccagcgccaccaccgcctacctgcagatcaacaacctgaagaccgaggacaccgccacctacttctgcgccaggggcgagatctactacggctacgacggcggcttcgcctactggggccagggcaccctggtgaccgtgagcgccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagccaccaacttcagcctgctgaagcaggccggcgacgtggaggagaaccccggccccatggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaaggcagcggcgaaggccgcggcagcctgctgacctgcggcgatgtggaagaaaacccgggccccatgtacagaatgcagctgctgagctgcatcgccctgagcctggccctggtgaccaacagcggcatccacgtgttcatcctgggctgcttcagcgccggcctgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgcttcctgctggagctgcaggtgatcagcctggagagcggcgacgccagcatccacgacaccgtggagaacctgatcatcctggccaacaacagcctgagcagcaacggcaacgtgaccgagagcggctgcaaggagtgcgaggagctggaggagaagaacatcaaggagttcctgcagagcttcgtgcacatcgtgcagatgttcatcaacaccagctccggcggcggctccggcggcggcggctccggcggcggcggctccggcggcggcggctccggcggcggctccctgcagatcacctgccccccccccatgagcgtggagcacgccgacatctgggtgaagagctacagcctgtacagcagagagagatacatctgcaacagcggcttcaagagaaaggccggcaccagcagcctgaccgagtgcgtgctgaacaaggccaccaacgtggcccactggaccacccccagcctgaagtgcatcagaaccaccacccccgcccccaggccccccacccccgcccccaccatcgccagccagcccctgagcctgaggcccgaggcctgcaggcccgccgccggcggcgccgtgcacaccaggggcctggacttcgcctgcgacgtggctatctccacgtccactgtcctgctgtgtgggctgagcgctgtgtctctcctggcatgctactaagtttaaac